(19) United States
(12) Patent Application Publication
(10) Pub. No.: US 2023/0056189 A1
(43) Pub. Date: Feb. 23, 2023

(54) COMPOSITIONS AND METHODS FOR TREATMENT OF PATHOLOGIC PAIN ASSOCIATED WITH MALIGNANT GROWTH DISORDER

(71) Applicant: Duke University, Durham, NC (US)

(72) Inventors: Wolfgang Liedtke, Durham, NC (US); Ru-Rong Ji, Durham, NC (US)

(21) Appl. No.: 17/912,429

(22) PCT Filed: Mar. 18, 2021
(86) PCT No.: PCT/US21/22917

§ 371 Date: Sep. 16, 2022

Related U.S. Application Data
(60) Provisional application No. 62/991,651, filed on Mar. 19, 2020.

(51) Int. Cl. A61K 31/55; A61K 31/365; A61K 31/167; A61K 31/428; A61K 33/243; A61K 31/166; A61K 31/17; A61K 31/519; A61K 31/7068; A61K 31/475; A61K 31/337; A61K 31/7048; A61K 31/704; A61P 35/00

(52) U.S. Cl. CPC A61K 31/55; A61K 31/365; A61K 31/167; A61K 31/428; A61K 33/243; A61K 31/166; A61K 31/17; A61K 31/519; A61K 31/7068; A61K 31/475; A61K 31/337; A61K 31/7048; A61K 31/704; A61P 35/00

(57) ABSTRACT

The present disclosure describes, in part, compositions and methods for treating pathologic pains associated with malignant growth disorders by administering a therapeutically effective amount of K+/Cl-cotransporter (Kcc2/KCC2) gene expression enhancer to a subject in need.

### CROSS REFERENCE TO RELATED APPLICATIONS

This application claims benefit of priority to U.S. Provisional Patent Application No. 62/991,651, filed Mar. 19, 2020, the contents of which is hereby incorporated by reference in its entirety.

### FIELD

The present disclosure generally relates to pain treatment. More specifically, the present disclosure relates to compositions and methods for the treatment of pathologic pains associated with malignant growth disorders.

### FEDERAL FUNDING LEGEND

This invention was made with government support under Federal Grant No. NS066307 awarded by the National Institute of Health (NIH). The Federal Government has certain rights to this invention.

### BACKGROUND

Inhibitory γ-aminobutyric acid (GABA)-ergic neurotransmission is of fundamental relevance for the adult vertebrate central nervous system (CNS) and requires low chloride ion concentration in neurons. In the mature vertebrate CNS, GABA acts primarily as an inhibitory neurotransmitter and is critical for normal CNS functioning (Fiumelli, et al., 2005, Neuron 48: 773-786; Ganguly, et al., 2001, Cell 105: 521-532). In chronic pain, GABA-ergic transmission is compromised, causing circuit malfunction and disrupting inhibitory networks (Coull et al., 2003, Nature 424: 938-942; Delpire & Mount, 2002, Annual Review of Physiology 64: 803 843).

In the adult vertebrate CNS, the K⁺/Cl⁻ cotransporter KCC2 is expressed exclusively in neurons. KCC2 continuously extrudes chloride ions, thus ensuring that intracellular levels of chloride ions remain low as required for inhibitory GABA-ergic neurotransmission (Cancedda, et al., 2007, J Neurosci 27: 5224-5235; Zhu, et al., 2008, Epilepsy Research 79: 201-212; Agez, et al., 2017, Scientific Reports 7: 16452). In chronic pathologic pain, KCC2 expression is attenuated in the primary sensory gate in spinal cord dorsal horn (SCDH) neurons. This key pathophysiological mechanism contributes to an excitation/inhibition imbalance because it corrupts inhibitory neurotransmission leading to inhibitory circuit malfunction (Doyon, et al., 2013, Expert Review of Neurotherapeutics 13: 469-471; Kahle, et al., 2014, JAMA Neurology 71: 640-645; Braz, et al., 2017, Prog Brain Res 231: 87-105). Notably, there is no “back-up” protein that can rescue the KCC2 expression deficit.

Therefore, there is a need to discover new approaches for the restoration of physiologic GABA-ergic transmission, which would increase the basic understanding of these sensory disorders to address the unmet medical need of chronic pain with safer and more effective alternatives to opioids for chronic pain. The present disclosure fulfills this long-standing need.

### SUMMARY

The present disclosure provides, in part, identification of compounds that enhance K⁺/Cl-cotransporter (Kcc2/KCC2; Kcc2—rodent gene; KCC2—human gene) gene expression and are useful in treatment of pathologic pains. The identified compounds inhibit the function and/or activity of GSK3β in neurons. The present disclosure further provides methods of treating pathologic pains associated with malignant growth disorders using the compounds identified herein and kits comprising the same.

Accordingly, one aspect of the present disclosure provides a method of treating a pathologic pain associated with a malignant growth disorder in a subject in need thereof. Such method comprises administering to the subject a therapeutically effective amount of a K+/Cl-cotransporter (Kcc2/KCC2) gene expression enhancer. In one embodiment, the Kcc2/KCC2 gene expression enhancer is a GSK3β inhibitor. In one embodiment, the GSK3β inhibitor inhibits the activity and/or function of GSK3β in a neuronal cell.

In one embodiment, the Kcc2/KCC2 gene expression enhancer is a compound comprising the general formula (I) (termed NSC180515):

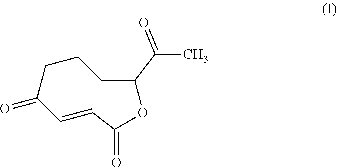

or a pharmaceutically acceptable salt, solvate, hydrate, prodrug, or derivative thereof.

In one embodiment, the Kcc2/KCC2 gene expression enhancer is a compound comprising the general formula (II) (termed NSC79456):

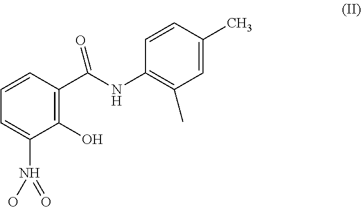

or a pharmaceutically acceptable salt, solvate, hydrate, prodrug, or derivative thereof.

In one embodiment, the Kcc2/KCC2 gene expression enhancer is a compound comprising the general formula (III) (termed NSC33006):

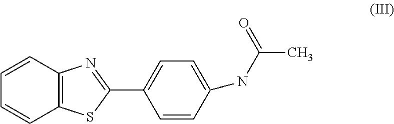

or a pharmaceutically acceptable salt, solvate, hydrate, prodrug, or derivative thereof.

In one embodiment, the Kcc2/KCC2 gene expression enhancer is a compound comprising the general formula (IV) (termed Kenpaullone (KP)):

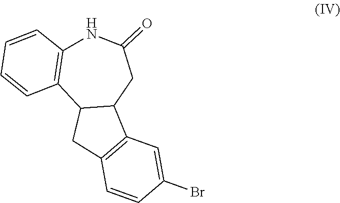

or a pharmaceutically acceptable salt, solvate, hydrate, prodrug, or derivative thereof.

Another aspect of the present disclosure provides a method of reducing intracellular chloride ion levels in a central nervous system cell in a subject in need thereof. Such method comprises administering to the subject a therapeutically effective amount of a K+/Cl-cotransporter (Kcc2) gene expression enhancer. In one embodiment, the Kcc2 gene expression enhancer is a GSK3β inhibitor. In one embodiment, the GSK3β inhibitor inhibits the activity and/or function of GSK3β in a neuronal cell.

Still another aspect of the present disclosure provides a method of increasing chloride ion efflux in a central nervous system cell in a subject in need thereof. Such method comprises administering to the subject a therapeutically effective amount of a K+/Cl-cotransporter (Kcc2) gene expression enhancer. In one embodiment, the Kcc2 gene expression enhancer is a GSK3βinhibitor. In one embodiment, the GSK3β inhibitor inhibits the activity and/or function of GSK3β in a neuronal cell.

Yet another aspect of the present disclosure provides a method of restoring KCC2 function thereby treating a neurologic and mental health condition in a subject in need thereof. Such method comprises administering to the subject a therapeutically effective amount of a K+/Cl-cotransporter (Kcc2) gene expression enhancer. In one embodiment, the Kcc2 gene expression enhancer is a GSK3β inhibitor. In one embodiment, the GSK3β inhibitor inhibits the activity and/or function of GSK3β in a neuronal cell.

In the methods described in all relevant aspects of the disclosure, by way of non-limiting example, the Kcc2/KCC2 gene expression enhancer is a compound comprising the above-mentioned general formula (I) (termed NSC180515), or a pharmaceutically acceptable salt, solvate, hydrate, prodrug, or derivative thereof.

By way of non-limiting example, the Kcc2/KCC2 gene expression enhancer is a compound comprising the above-mentioned general formula (II) (termed NSC79456), or a pharmaceutically acceptable salt, solvate, hydrate, prodrug, or derivative thereof.

By way of non-limiting example, the Kcc2/KCC2 gene expression enhancer is a compound comprising the general formula (III) (termed NSC33006), or a pharmaceutically acceptable salt, solvate, hydrate, prodrug, or derivative thereof.

By way of non-limiting example, the Kcc2/KCC2 gene expression enhancer is a compound comprising the general formula (IV) (termed Kenpaullone (KP)), or a pharmaceutically acceptable salt, solvate, hydrate, prodrug, or derivative thereof.

Still yet another aspect of the present disclosure provides a kit for treating a pathologic pain. Such kit comprises a therapeutically effective amount of a K+/Cl-cotransporter (Kcc2) gene expression enhancer, a means of administering the enhancer, and instructions for use.

Still yet another aspect of the present disclosure provides a kit for treating a neurologic and mental health condition. Such kit comprises a therapeutically effective amount of a K+/Cl-cotransporter (Kcc2) gene expression enhancer, a means of administering the enhancer, and instructions for use.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

In order to better understand the subject matter that is disclosed herein and to exemplify how it may be carried out in practice, embodiments will now be described, by way of non-limiting example only, with reference to the accompanying drawings, in which:

FIG. 1 is a schematic of compound screening for enhancers of Kcc2 gene expression in primary mouse cortical neurons. Four compound “winners” are identified in the bottom panel.

FIGS. 2A-2F show that kenpaullone (KP) enhances Kcc2/KCC2 gene expression and function in rat and human primary cortical neurons. FIG. 2A is a graph illustrating dose-dependent increase in Kcc2 mRNA expression in primary rat cortical neurons after KP treatment, also reflected by increased protein expression as shown by KCC2 immuno-label (micrographs on the right). FIG. 2B is a graph illustrating reduction of neuronal [Cl−]I, measured with clomeleon, after KP treatment in primary rat cortical neurons. FIG. 2C is a graph illustrating that add-on treatment with KCC2-transport blocker (VU0240551) leads to an increased [Cl-]i for both vehicle-treated and KP-treated groups. FIG. 2D is a graph illustrating dose-dependent increase in KCC2 mRNA expression in primary human cortical neurons after KP treatment. FIG. 2E is a series of confocal images at DIV 10 immuno-labelled for KCC2 and synaptophysin after vehicle or KP-treatment. FIG. 2F is a graph illustrating increased KCC2 (left-hand) and synaptophysin (right-hand) expression after KP treatment, relative to vehicle treatment, in primary human cortical neurons.

FIG. 3A is a graph showing no difference in neuronal chloride levels after KP treatment vs. vehicle treatment at the 10 min and 20 min time-points in primary rat cortical neurons. FIG. 3B is a graph showing clomeleon-based measurement of [Cl-]i in rat primary cortical neurons. FIG. 3C is a graph showing rat primary cortical neurons transfected with renilla luciferase and driven by Kcc2 promoter.

FIGS. 4A-4D illustrate that kenpaullone (KP) is analgesic in mouse nerve constriction injury and bone cancer pain. FIG. 4A is a bar diagram demonstrating dose-dependent analgesic effects of KP for sensitized mechanical withdrawal in mouse nerve constriction injury pain, wherein the bottom panel is an overview of timeline of systemic injection and behavioral metrics. FIG. 4B is a bar diagram demonstrating analgesic effects of KP for sensitized mechanical withdrawal in mouse bone cancer pain, wherein the bottom panel is an overview of timeline of systemic injection and metrics. FIG. 4C is a bar diagram demonstrating that KP reduces mechanical allodynia in neuropathic pain following nerve constriction injury, wherein the bottom panel is an overview of timeline of nerve constriction (CCI). FIG. 4D is a bar diagram demonstrating that co-application of KP and VU0240551 blocks the central analgesic effects of KP, wherein the bottom panel is an overview of timeline of nerve constriction (CCI).

FIGS. 5A-5C illustrate the effects of kenpaullone (KP) on lytic bone lesions, Rotarod-performance and conditioned place preference (CPP). FIG. 5A shows that KP did not contain growth of mouse LLC lung tumor cells infused into the femur (top images) and that osteolytic damage to femur was not contained at any time point (bottom bar diagram). FIG. 5B is a bar diagram showing the means of elapsed time on the rotarod on different days. FIG. 5C shows timeline for CPP assay (top panel). No significant difference in CPP scores between KP and vehicle was observed (bottom graph).

FIGS. 6A-6D illustrate that kenpaullone (KP) re-normalizes E_{GABA} in spinal cord dorsal horn by increasing Kcc2 gene expression/function. FIG. 6A shows Lamina-I/II area of spinal cord dorsal horn (SCDH) (top left image), Lamina-I/II area before and after laser capture microdissection (top right images), and the Kcc2 mRNA expression in the SCDH of KP-treated vs. vehicle-treated mice after nerve constriction injury (PSNL) (bottom bar diagram). FIG. 6B shows increased KCC2 protein expression in SCDH after KP treatment compared to vehicle control in PSNL (Left bar diagram), and representative KCC2 immuno-staining of the SCDH in PSNL (right images). FIG. 6C shows potent behavioral sensitization of juvenile mice after PSNL and its almost complete behavioral recovery after treatment with KP. FIG. 6D shows a representative I-V plot (left), and quantification of E_{GABA} (right).

FIGS. 7A-7B are electrophysiological recordings from spinal cord dorsal horn. FIG. 7A shows current responses of layer-II neurons to a voltage ramp from +8 to −92 mV (shown in FIG. 7B) in control (grey traces, obtained before GABA puff), or at the end of a puff of GABA (black trace) in sham, PSNL+vehicle and PSNL+KP groups.

FIG. 8 is a bar graph showing GSK3-inhibitors increased Kcc2 mRNA expression in a dose-dependent manner as measured by RT-qPCR, whereas several CDK-inhibitors did not increase Kcc2 mRNA expression, or even reduced it.

FIGS. 9A-9D illustrate cellular mechanism of action of kenpaullone (KP) in central neurons. FIG. 9A shows identification of proteins that bind to KP in rat primary cortical neurons using DARTS methodology (top), and that KP binding to GSK3β is independent of detergent treatment of the protein sample prepared from the neuronal culture (bottom). FIG. 9B shows the results of phosphoproteomics assays (top), and the structure of human δ-cat (CTNND2) (bottom). FIG. 9C shows representative immuno-labeling of βrn-tubulin (neuronal isoform of β-tubulin) and δ-cat before and after KP treatment in rat primary cortical neurons (top), and that KP significantly increases δ-cat nuclear translocation (bottom). FIG. 9D shows representative immuno-labeling of β_{III}-tubulin and δ-cat before and after KP treatment in differentiated N2a mouse neural cells transfected with either hu-δ-cat (WT), hu-δ-cat (S276A) or control vector (top), and that KP significantly enhances nuclear transfer of δ-cat when transfected with δ-cat (WT) (bottom).

FIGS. 10A-10C illustrate that kenpaullone (KP) enhances neuronal differentiation and facilitates β-catenin translocation to the nucleus. FIG. 10A shows neuronalization of N2a cultured cells in response to KP. FIG. 10B shows representative immuno-labeling of neuronal β_{III}-tubulin (green) and β-catenin (red) before (left-hand panels) and after KP treatment (right-hand panels) in primary cortical neurons from rat. FIG. 10C shows that KP significantly increases β-catenin translocation into the nucleus.

FIGS. 11A-11B illustrate that kenpaullone (KP) regulates Kcc2 promoter activity via δ-cat and two Kaiso sites. FIG. 11A shows structure of mouse Kcc2 gene encompassing 2.5 kb surrounding the transcription start site (TSS; +1) (top), and chromatin immuno-precipitation (ChIP) using anti-δ-cat antibody in rat primary cortical neurons (bottom). FIG. 11B shows mouse Kcc2 promoter constructs, Kaiso1, -2 were deleted and a ΔK1/K2 construct was built devoid of both sites (top), and Luciferase (LUC) activity of Kcc2 promoter constructs in N2a cells with neuronal differentiation (bottom).

FIGS. 12A-12B illustrate that kenpaullone (KP) increases β-catenin binding to Kcc2 promoter, relevance TCF site. FIG. 12A shows Structure of mouse-Kcc2 gene encompassing 2.5 kb surrounding the TSS (+1) (top), and chromatin immuno-precipitation using anti-β-cat antibody in primary rat cortical neurons (bottom). FIG. 12B shows mouse Kcc2 promoter constructs (top), and that KP did not increase promoter activity for any of the constructs (bottom).

FIG. 13 is a schematic showing the analgesic mechanism of action of kenpaullone (KP).

FIGS. 14A-14C illustrate that paclitaxel and kenpaullone (KP) function synergistically in their impact on cell viability of two commonly used breast cancer cell lines (FIGS. 14A-14B) and a melanoma cell line (FIG. 14C).

FIGS. 15A-15B illustrate effects of targeted overexpression of δ-catenin in neuronal-neuroblastoma cell line N2a. FIG. 15A shows significantly reduced % BrdU incorporation in cultures with δ-cat targeted expression. FIG. 15B shows significantly enhanced neuronal process extension in cultures with δ-cat targeted expression.

### DETAILED DESCRIPTION

For the purposes of promoting an understanding of the principles of the present disclosure, reference will now be made to preferred embodiments and specific language will be used to describe the same. It will nevertheless be understood that no limitation of the scope of the disclosure is thereby intended, such alteration and further modifications of the disclosure as illustrated herein, being contemplated as would normally occur to one skilled in the art to which the disclosure relates.

Section headings as used in this section and the entire disclosure herein are merely for organizational purposes and are not intended to be limiting.

### Definitions

Unless otherwise defined, all technical and scientific terms used herein have the same meaning as commonly understood by one of ordinary skill in the art. In case of conflict, the present document, including definitions, will control. Preferred methods and materials are described below, although methods and materials similar or equivalent to those described herein can be used in practice or testing of the present disclosure. All publications, patent applications, patents and other references mentioned herein are incorporated by reference in their entirety. The materials, methods, and examples disclosed herein are illustrative only and not intended to be limiting. As used herein, the following terms have the meanings ascribed to them unless specified otherwise.

Articles “a” and “an” are used herein to refer to one or to more than one (i.e., at least one) of the grammatical object of the article. By way of example, “an element” means at least one element and can include more than one element.

“About” is used herein to provide flexibility to a numerical range endpoint by providing that a given value may be “slightly above” or “slightly below” the endpoint without affecting the desired result.

As used herein, the term “including,” “comprising,” or “having,” and variations thereof, is meant to encompass the elements listed thereafter and equivalents thereof as well as additional elements. As used herein, “and/or” refers to and encompasses any and all possible combinations of one or more of the associated listed items, as well as the lack of combinations where interpreted in the alternative (“or”).

As used herein, the transitional phrase “consisting essentially of” (and grammatical variants) is to be interpreted as encompassing the recited materials or steps “and those that do not materially affect the basic and novel characteristic(s)” of the claimed invention. Thus, the term “consisting essentially of” as used herein should not be interpreted as equivalent to “comprising.”

Moreover, the present disclosure also contemplates that in some embodiments, any feature or combination of features set forth herein can be excluded or omitted. To illustrate, if the specification states that a complex comprises components A, B and C, it is specifically intended that any of A, B or C, or a combination thereof, can be omitted and disclaimed singularly or in any combination.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure.

As used herein, “treatment,” “therapy” and/or “therapy regimen” refer to the clinical intervention made in response to a disease, disorder or physiological condition manifested by a patient or to which a patient may be susceptible. The aim of treatment includes the alleviation or prevention of symptoms, slowing or stopping the progression or worsening of a disease, disorder, or condition and/or the remission of the disease, disorder or condition.

As used herein, the term “inhibit” or “inhibiting” refers to reduction in the amount, levels, density, turnover, association, dissociation, activity, signaling, or any other feature associated with the protein.

As used herein, “administration” of a disclosed compound encompasses the delivery to a subject of a compound as described herein, or a prodrug or other pharmaceutically acceptable derivative thereof, using any suitable formulation or route of administration, as discussed herein.

The term “effective amount” or “therapeutically effective amount” refers to an amount sufficient to effect beneficial or desirable biological and/or clinical results including, but not limited to, disease treatment, as illustrated below. In some embodiments, the amount is that effective for detectable reduction of pain. In some embodiments, the amount is that effective for alleviating, reducing or eliminating a pathologic pain.

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, e.g., the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

As used herein, the terms “disease” and “disorder” are interchangeable, including, but not limited to, any abnormal condition and/or disorder of a structure or a function that affects a part of an organism. It may be caused by an external factor, such as an infectious disease, or by internal dysfunctions, such as cancer, cancer metastasis, and the like.

As is known in the art, a cancer is generally considered as uncontrolled cell growth. The methods of the present invention can be used to treat any cancer, and any metastases thereof, including, but not limited to, carcinoma, lymphoma, blastoma, sarcoma, and leukemia. More particular examples of such cancers include breast cancer, bone cancer, prostate cancer, colon cancer, squamous cell cancer, small-cell lung cancer, non-small cell lung cancer, ovarian cancer, cervical cancer, gastrointestinal cancer, pancreatic cancer, glioblastoma, liver cancer, bladder cancer, hepatoma, colorectal cancer, uterine cervical cancer, endometrial carcinoma, salivary gland carcinoma, mesothelioma, kidney cancer, vulvar cancer, pancreatic cancer, thyroid cancer, hepatic carcinoma, skin cancer, melanoma, brain cancer, neuroblastoma, myeloma, various types of head and neck cancer, acute lymphoblastic leukemia, acute myeloid leukemia, Ewing sarcoma and peripheral neuroepithelioma.

As used herein, the term “pain” refers to the basic bodily sensation induced by a noxious stimulus, received by naked nerve endings, characterized by physical discomfort (e.g., pricking, throbbing, aching, etc.) and typically leading to an evasive action by the individual. The term pain also includes pathologic pain. As used herein, the terms “pathologic pain” and “pathological pain” can be used interchangeably and refer to that pain which is characterized by an amplified response to normally innocuous stimuli, and an amplified response to acute pain. Pathologic pain can be either acute or chronic. As used herein, the term pain further includes chronic and acute neuropathic pain. The terms “neuropathic pain” or “neurogenic pain” can be used interchangeable and refer to pain that arises from direct stimulation of nervous tissue itself, central or peripheral and can persist in the absence of stimulus. The sensations that characterize neuropathic pain vary and are often multiple and include burning, gnawing, aching, and shooting (Rooper and Brown, (2005) Adams and Victor's Principles of Neurology, 8^{th} ed., NY, McGraw-Hill). These damaged nerve fibers send incorrect signals to other pain centers. The impact of nerve fiber injury includes a change in nerve function both at the site of injury and areas around the injury. Chronic neuropathic pain often seems to have no obvious cause, however, some common causes may include, but are not limited to, alcoholism, amputation, back, leg and hip problems, chemotherapy, diabetes, facial nerve problems, HIV infection or AIDS, multiple sclerosis, shingles, and spine surgery. For example, one example of neuropathic pain is phantom limb syndrome, which occurs when an arm or leg has been removed because of illness or injury, but the brain still gets pain messages from the nerves that originally carried impulses from the missing limb.

As used herein, the term “subject” and “patient” are used interchangeably herein and refer to both human and nonhuman animals. The term “nonhuman animals” of the disclosure includes all vertebrates, e.g., mammals and non-mammals, such as nonhuman primates, sheep, dog, cat, horse, cow, chickens, amphibians, reptiles, and the like. The methods and compositions disclosed herein can be used on a sample either in vitro (for example, on isolated cells or tissues) or in vivo in a subject (i.e. living organism, such as a patient). In some embodiments, the subject comprises a subject suffering from pain. In other embodiments, the subject is suffering from pain as a result of a disease, such as a cancer.

Inhibitory GABA-ergic neurotransmission is fundamental for the adult vertebrate central nervous system and requires low chloride ion concentration in neurons. This basic ionic-homeostatic mechanism relies on expression and function of KCC2, a neuroprotective ionic transporter that extrudes neuronal chloride. Importantly, no other transporter can rescue KCC2 deficit, and attenuated expression of KCC2 is strongly associated with circuit malfunction in chronic pain, epilepsy, neuro-degeneration, neuro-trauma, and other neuro-psychiatric illnesses. To isolate Kcc2/KCC2 gene expression-enhancing compounds, 1057 cell growth-regulating compounds were screened in cultured primary cortical neurons. Several compounds including kenpaullone (KP) were identified. KP enhanced Kcc2/KCC2 expression and function in cultured rodent and human neurons by inhibiting GSK3β. KP effectively reduced pathologic pain in preclinical mouse models of nerve constriction injury and bone cancer. In nerve-injury pain, KP restored Kcc2 expression and GABA-evoked chloride reversal potential in the spinal cord dorsal horn. δ-catenin, a phosphorylation-target of GSK3β in neurons, activated the Kcc2 promoter via Kaiso transcription factor. Validating this new pathway in-vivo, transient spinal over-expression of δ-catenin mimicked KP analgesia. With relevance for pathologic pain, the discoveries of a newly repurposed compound that enhanced Kcc2 gene expression enabled re-normalization of disrupted inhibitory neurotransmission through genetic re-programming.

In one aspect, the present disclosure provides a method of treating a pathologic pain associated with a malignant growth disorder in a subject in need thereof. Such method comprises administering to the subject a therapeutically effective amount of a K+/Cl-cotransporter (Kcc2/KCC2) gene expression enhancer. In one embodiment, the Kcc2 gene expression enhancer is a GSK3β inhibitor. In one embodiment, the GSK3β inhibitor inhibits the activity and/or function of GSK3β in a neuronal cell.

In the adult vertebrate central nervous system (CNS), KCC2 is expressed exclusively in neurons. KCC2 continuously extrudes chloride ions, thus ensuring that intracellular levels of chloride ions remain low as required for inhibitory GABA-ergic neurotransmission. In chronic pathologic pain, KCC2 expression is attenuated in the primary sensory gate in spinal cord dorsal horn (SCDH) neurons. This key pathophysiological mechanism contributes to an excitation/inhibition imbalance because it corrupts inhibitory neurotransmission leading to inhibitory circuit malfunction. Thus, it is expected that if Kcc2/KCC2 gene expression (Kcc2—rodent gene; KCC2—human gene) could be boosted, then inhibitory transmission could be normalized for relief of chronic pain. An unbiased screen of cell growth-regulating compounds were conducted to identify candidates upregulating gene expression of Kcc2/KCC2, which would in turn lower neuronal chloride levels and subsequently provide pain relief.

In one embodiment, the Kcc2 gene expression enhancer is a compound comprising the general formula (I) (termed NSC180515):

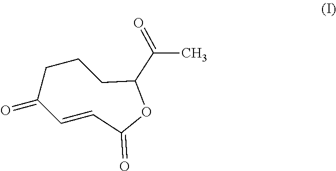

or a pharmaceutically acceptable salt, solvate, hydrate, prodrug, or derivative thereof.

In one embodiment, the Kcc2 gene expression enhancer is a compound comprising the general formula (II) (termed NSC79456):

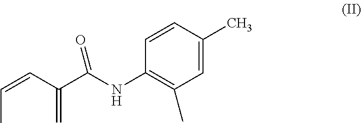

or a pharmaceutically acceptable salt, solvate, hydrate, prodrug, or derivative thereof.

In one embodiment, the Kcc2 gene expression enhancer is a compound comprising the general formula (III) (termed NSC33006):

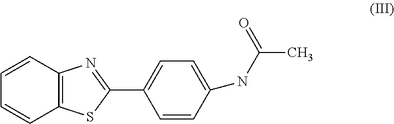

or a pharmaceutically acceptable salt, solvate, hydrate, prodrug, or derivative thereof.

In one embodiment, the Kcc2 gene expression enhancer is a compound comprising the general formula (IV) (termed Kenpaullone):

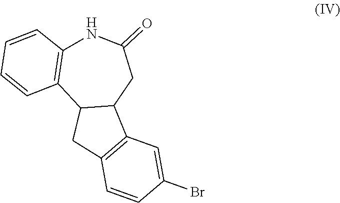

or a pharmaceutically acceptable salt, solvate, hydrate, prodrug, or derivative thereof.

The above compounds were identified as Kcc2/KCC2 gene expression enhancers, among which kenpaullone (KP) functioned as an analgesic in preclinical mouse models. The data suggests that the cellular mechanism of action of KP in neurons was based on its GSK3β-inhibitory function which enhanced Kcc2/KCC2 gene expression.

In one embodiment, the malignant growth disorder is a cancer. By way of non-limiting example, the cancer is bone cancer. The bone cancer may be caused by skeletal cancer or other malignant cells. By way of non-limiting example, the cancer is a blood cancer. The blood cancer may be multiple myeloma, lymphoma, or leukemia.

In one embodiment, at least one additional therapeutic agent may be further administered to the subject in need thereof. By way of non-limiting example, the additional therapeutic agent may be a pain killer, a steroid, an anti-cancer drug, a muscle relaxant, an anti-anxiety drug, an antidepressant, an anticonvulsant, an antimetabolite, an antimicrotubule agent, a topoisomerase inhibitor, a cytotoxic agent, a checkpoint inhibitor, or a combination thereof.

When used to treat or prevent such diseases (e.g., pain), the compounds described herein may be administered singly, as mixtures of one or more compounds or in mixture or combination with other agents useful for treating such diseases and/or the symptoms associated with such diseases. The compounds may also be administered in mixture or in combination with agents useful to treat other disorders or maladies, such as other analgesics (e.g., NTHES, acetaminophen, topical analgesics (e.g., lidocaine patches/creams), steroids, anticonvulsants, antidepressants, etc.), anticancer/chemotherapeutic drugs (e.g., alkylating agents (e.g., cisplatin, chlorambucil, procarbazine, carmustine, etc.), antimetabolites (e.g., methotrexate, cytarabine, gemcitabine, etc.), antimicrotubule agents (e.g., vinblastine, paclitaxel, etc.), topoisomerase inhibitors (e.g., etoposide, doxorubicin, etc.), cytotoxic agents (e.g., bleomycin, mitomycin, etc.), checkpoint inhibitors (e.g., anti-PD1, anti-CTLA4, etc.), and combinations thereof. The compounds may be administered in the form of compounds per se, or as pharmaceutical compositions comprising a compound.

In one embodiment, the compounds may be administered in combination with a therapeutic treatment modality. By way of non-limiting example, the therapeutic treatment modality may be a surgery, a radiation therapy, a cryosurgery, a thermotherapy, or a combination thereof.

Pharmaceutical compositions comprising the compound(s) may be manufactured by means of conventional mixing, dissolving, granulating, dragee-making levigating, emulsifying, encapsulating, entrapping or lyophilization processes. The compositions may be formulated in conventional manner using one or more physiologically acceptable carriers, diluents, excipients or auxiliaries which facilitate processing of the compounds into preparations which can be used pharmaceutically. The exact nature of the carrier, diluent, excipient or auxiliary will depend upon the desired use for the composition, and may range from being suitable or acceptable for veterinary uses to being suitable or acceptable for human use. The composition may optionally include one or more additional compounds.

The compounds may be formulated in the pharmaceutical composition per se, or in the form of a hydrate, solvate, N-oxide or pharmaceutically acceptable salt, as previously described. Typically, such salts are more soluble in aqueous solutions than the corresponding free acids and bases, but salts having lower solubility than the corresponding free acids and bases may also be formed.

In one embodiment, the compounds described above and herein may be administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually. By way of non-limiting example, the compounds may be administered intraperitoneally.

Pharmaceutical compositions may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the compound(s) may be formulated as solutions, gels, ointments, creams, suspensions, etc. as are well-known in the art. Systemic formulations include those designed for administration by injection, e.g., subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, e.g., in ampules or in multidose containers, and may contain added preservatives. Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (e.g., pregelatinised maize starch, polyvinylpyrrolidone or hydroxypropyl methylcellulose); fillers (e.g., lactose, microcrystalline cellulose or calcium hydrogen phosphate); lubricants (e.g., magnesium stearate, talc or silica); disintegrants (e.g., potato starch or sodium starch glycolate); or wetting agents (e.g., sodium lauryl sulfate). The tablets may be coated by methods well known in the art with, for example, sugars, films or enteric coatings.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (e.g., sorbitol syrup, cellulose derivatives or hydrogenated edible fats); emulsifying agents (e.g., lecithin or acacia); non-aqueous vehicles (e.g., almond oil, oily esters, ethyl alcohol, Cremophore™ or fractionated vegetable oils); and preservatives (e.g., methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the compound, as is well known. For buccal administration, the compositions may take the form of tablets or lozenges formulated in conventional manner. For rectal and vaginal routes of administration, the compound(s) may be formulated as solutions (for retention enemas) suppositories or ointments containing conventional suppository bases such as cocoa butter or other glycerides.

For nasal administration or administration by inhalation or insufflation, the compound(s) can be conveniently delivered in the form of an aerosol spray from pressurized packs or a nebulizer with the use of a suitable propellant, e.g., dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

For ocular administration, the compound(s) may be formulated as a solution, emulsion, suspension, etc. suitable for administration to the eye. A variety of vehicles suitable for administering compounds to the eye are known in the art.

For prolonged delivery, the compound(s) can be formulated as a depot preparation for administration by implantation or intramuscular injection. The compound(s) may be formulated with suitable polymeric or hydrophobic materials (e.g., as an emulsion in an acceptable oil) or ion exchange resins, or as sparingly soluble derivatives, e.g., as a sparingly soluble salt. Alternatively, transdermal delivery systems manufactured as an adhesive disc or patch which slowly releases the compound(s) for percutaneous absorption may be used. To this end, permeation enhancers may be used to facilitate transdermal penetration of the compound(s).

Alternatively, other pharmaceutical delivery systems may be employed. Liposomes and emulsions are well-known examples of delivery vehicles that may be used to deliver compound(s). Certain organic solvents such as dimethyl sulfoxide (DMSO) may also be employed, although usually at the cost of greater toxicity.

The pharmaceutical compositions may, if desired, be presented in a pack or dispenser device which may contain one or more unit dosage forms containing the compound(s). The pack may, for example, comprise metal or plastic foil, such as a blister pack. The pack or dispenser device may be accompanied by instructions for administration.

The compound(s) described herein, or compositions thereof, will generally be used in an amount effective to achieve the intended result, for example in an amount effective to treat or prevent the particular disease being treated. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. Therapeutic benefit also generally includes halting or slowing the progression of the disease, regardless of whether improvement is realized.

The amount of compound(s) administered will depend upon a variety of factors, including, for example, the particular indication being treated, the mode of administration, whether the desired benefit is prophylactic or therapeutic, the severity of the indication being treated and the age and weight of the patient, the bioavailability of the particular compound(s) the conversation rate and efficiency into active drug compound under the selected route of administration, etc.

Determination of an effective dosage of compound(s) for a particular use and mode of administration is well within the capabilities of those skilled in the art. Effective dosages may be estimated initially from in vitro activity and metabolism assays. For example, an initial dosage of compound for use in animals may be formulated to achieve a circulating blood or serum concentration of the metabolite active compound that is at or above an IC₅₀ of the particular compound as measured in as in vitro assay. Calculating dosages to achieve such circulating blood or serum concentrations taking into account the bioavailability of the particular compound via the desired route of administration is well within the capabilities of skilled artisans. Initial dosages of compound can also be estimated from in vivo data, such as animal models. Animal models useful for testing the efficacy of the active metabolites to treat or prevent the various diseases described above are well-known in the art. Animal models suitable for testing the bioavailability and/or metabolism of compounds into active metabolites are also well-known. Ordinarily skilled artisans can routinely adapt such information to determine dosages of particular compounds suitable for human administration.

Dosage amounts will typically be in the range of from about 0.0001 mg/kg/day, 0.001 mg/kg/day or 0.01 mg/kg/day to about 100 mg/kg/day, but may be higher or lower, depending upon, among other factors, the activity of the active compound, the bioavailability of the compound, its metabolism kinetics and other pharmacokinetic properties, the mode of administration and various other factors, discussed above. Dosage amount and interval may be adjusted individually to provide plasma levels of the compound(s) and/or active metabolite compound(s) which are sufficient to maintain therapeutic or prophylactic effect. For example, the compounds may be administered once per week, several times per week (e.g., every other day), once per day or multiple times per day, depending upon, among other things, the mode of administration, the specific indication being treated and the judgment of the prescribing physician. In cases of local administration or selective uptake, such as local topical administration, the effective local concentration of compound(s) and/or active metabolite compound(s) may not be related to plasma concentration. Skilled artisans will be able to optimize effective dosages without undue experimentation.

In another aspect, the present disclosure provides a method of reducing intracellular chloride ion levels in a central nervous system cell in a subject in need thereof. Such method comprises administering to the subject a therapeutically effective amount of a K+/Cl-cotransporter (Kcc2/KCC2) gene expression enhancer. In one embodiment, the Kcc2/KCC2 gene expression enhancer is a GSK3β inhibitor. In one embodiment, the GSK3β inhibitor inhibits the activity and/or function of GSK3β in a neuronal cell.

In one embodiment, the Kcc2/KCC2 gene expression enhancer is a compound comprising the above-mentioned general formula (I) (termed NSC180515), or a pharmaceutically acceptable salt, solvate, hydrate, prodrug, or derivative thereof. By way of non-limiting example, this compound is 2-Acetyl-2,3,4,5-tetrahydrooxonine-6,9-dione.

In one embodiment, the Kcc2/KCC2 gene expression enhancer is a compound comprising the above-mentioned general formula (II) (termed NSC79456), or a pharmaceutically acceptable salt, solvate, hydrate, prodrug, or derivative thereof. By way of non-limiting example, this compound is n-(2,4-Dimethylphenyl)-2-hydroxy-3-nitrobenzamide.

In one embodiment, the Kcc2/KCC2 gene expression enhancer is a compound comprising the above-mentioned general formula (III) (termed NSC33006), or a pharmaceutically acceptable salt, solvate, hydrate, prodrug, or derivative thereof. By way of non-limiting example, this compound is N-[4-(1,3-benzothiazol-2-yl)phenyl]acetamide.

In one embodiment, the Kcc2/KCC2 gene expression enhancer is a compound comprising the above-mentioned general formula (IV) (termed Kenpaullone), or a pharmaceutically acceptable salt, solvate, hydrate, prodrug, or derivative thereof. By way of non-limiting example, this compound is 9-bromo-7,12-dihydro-indolo [3,2-d] [1]benzazepin-6(5H)-one.

In one embodiment, the Kcc2/KCC2 gene expression enhancer is administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually. By way of non-limiting example, the Kcc2/KCC2 gene expression enhancer is administered intrathecally or intraperitoneally.

In still another aspect, the present disclosure provides a method of increasing chloride ion efflux in a central nervous system cell in a subject in need thereof. Such method comprises administering to the subject a therapeutically effective amount of a K+/Cl-cotransporter (Kcc2/KCC2) gene expression enhancer. In one embodiment, the GSK3β inhibitor inhibits the activity and/or function of GSK3β in a neuronal cell.

In one embodiment, the Kcc2/KCC2 gene expression enhancer is a compound comprising the above-mentioned general formula (I) (termed NSC180515), or a pharmaceutically acceptable salt, solvate, hydrate, prodrug, or derivative thereof. By way of non-limiting example, this compound is 2-Acetyl-2,3,4,5-tetrahydrooxonine-6,9-dione.

In one embodiment, the Kcc2/KCC2 gene expression enhancer is a compound comprising the above-mentioned general formula (II) (termed NSC79456), or a pharmaceutically acceptable salt, solvate, hydrate, prodrug, or derivative thereof. By way of non-limiting example, this compound is n-(2,4-Dimethylphenyl)-2-hydroxy-3-nitrobenzamide.

In one embodiment, the Kcc2/KCC2 gene expression enhancer is a compound comprising the above-mentioned general formula (III) (termed NSC33006), or a pharmaceutically acceptable salt, solvate, hydrate, prodrug, or derivative thereof. By way of non-limiting example, this compound is N-[4-(1,3-benzothiazol-2-yl)phenyl]acetamide.

In one embodiment, the Kcc2/KCC2 gene expression enhancer is a compound comprising the above-mentioned general formula (IV) (termed Kenpaullone), or a pharmaceutically acceptable salt, solvate, hydrate, prodrug, or derivative thereof. By way of non-limiting example, this compound is 9-bromo-7,12-dihydro-indolo [3,2-d] [1]benzazepin-6(5H)-one.

In one embodiment, the Kcc2/KCC2 gene expression enhancer is administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually. By way of non-limiting example, the Kcc2/KCC2 gene expression enhancer is administered intrathecally or intraperitoneally.

In yet another aspect, the present disclosure provides a method of restoring KCC2/KCC2 function thereby treating a neurologic and mental health condition in a subject in need thereof. Such method comprises administering to the subject a therapeutically effective amount of a K+/Cl-cotransporter (Kcc2/KCC2) gene expression enhancer. In one embodiment, the GSK3β inhibitor inhibits the activity and/or function of GSK3β in a neuronal cell.

In one embodiment, the neurologic and mental health condition is a neurodegeneration condition or a neurodevelopmental disorder. By way of non-limiting example, the neurodegeneration condition or neurodevelopmental disorder may be epilepsy, a traumatic spinal cord injury, a traumatic spinal cord or brain injury, Rett Syndrome, an Autism Spectrum Disorder, or Alzheimer's Disease.

In one embodiment, the Kcc2/KCC2 gene expression enhancer is a compound comprising the above-mentioned general formula (I) (termed NSC180515), or a pharmaceutically acceptable salt, solvate, hydrate, prodrug, or derivative thereof. By way of non-limiting example, this compound is 2-Acetyl-2,3,4,5-tetrahydrooxonine-6,9-dione.

In one embodiment, the Kcc2/KCC2 gene expression enhancer is a compound comprising the above-mentioned general formula (II) (termed NSC79456), or a pharmaceutically acceptable salt, solvate, hydrate, prodrug, or derivative thereof. By way of non-limiting example, this compound is n-(2,4-Dimethylphenyl)-2-hydroxy-3-nitrobenzamide.

In one embodiment, the Kcc2/KCC2 gene expression enhancer is a compound comprising the above-mentioned general formula (III) (termed NSC33006), or a pharmaceutically acceptable salt, solvate, hydrate, prodrug, or derivative thereof. By way of non-limiting example, this compound is N-[4-(1,3-benzothiazol-2-yl)phenyl]acetamide.

In one embodiment, the Kcc2/KCC2 gene expression enhancer is a compound comprising the above-mentioned general formula (IV) (termed Kenpaullone), or a pharmaceutically acceptable salt, solvate, hydrate, prodrug, or derivative thereof. By way of non-limiting example, this compound is 9-bromo-7,12-dihydro-indolo [3,2-d] [1]benzazepin-6(5H)-one.

In one embodiment, the Kcc2/KCC2 gene expression enhancer is administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually. By way of non-limiting example, the Kcc2/KCC2 gene expression enhancer is administered intrathecally or intraperitoneally.

In still yet another aspect, the present disclosure provides a kit for treating a pathologic pain. Such kit comprises a therapeutically effective amount of a K+/Cl-cotransporter (Kcc2/KCC2) gene expression enhancer, a means of administering the enhancer, and instructions for use.

In still yet another aspect, the present disclosure provides a kit for treating a neurologic and mental health condition, the kit comprising a therapeutically effective amount of a K+/Cl-cotransporter (Kcc2/KCC2) gene expression enhancer, a means of administering the enhancer, and instructions for use.

In the kits described in all relevant aspects of the disclosure, the Kcc2/KCC2 gene expression enhancer is a GSK3β inhibitor. In one embodiment, GSK3β inhibitor inhibits the activity and/or function of GSK3β in a neuronal cell.

In one embodiment, the Kcc2/KCC2 gene expression enhancer is Kenpaullone (9-bromo-7,12-dihydro-indolo [3,2-d] [1]benzazepin-6(5H)-one), NS C180515 (2-Acetyl-2,3,4,5-tetrahydrooxonine-6,9-di one), NSC79456 (n-(2,4-Dimethylphenyl)-2-hydroxy-3-nitrobenzamide), or NSC33006 (N-[4-(1,3-benzothiazol-2-yl)phenyl]acetamide) or a pharmaceutically acceptable salt, solvate, hydrate, prodrug, or derivative thereof.

In some embodiments, the kit is packaged in a container with a label affixed to the container or included in the package that describes use of the compounds described herein. Exemplary containers include, but are not limited to, a vessel, vial, tube, ampoule, bottle, flask, and the like. It is contemplated that the container is made from material well-known in the art, including, but not limited to, glass, polypropylene, polystyrene, and other plastics. In various aspects, the compounds are packaged in a unit dosage form. In various aspects, the kit contains a label and/or instructions that describes use of the contents for pain treatment.

The present disclosure characterizes a novel strategy to treat chronic pathologic pain. With a focus on enhanced expression of Kcc2/KCC2 in central neurons as an analgesic strategy, a compound was repurposed as a genomic reprogramming agent that reverted the expression of a key dysregulated gene. This effectively addressed a mechanism that underlies neural malplasticity in chronic pain: ineffective inhibitory neurotransmission in pain-relevant neural circuits caused by genomic dysregulation, such as the lack of expression of Kcc2/KCC2. Of practical/translational importance, it was found that kenpaullone (KP) is non-sedative and does not affect motor stamina, coordination, or choice behavior. Furthermore, KP was target-validated in human primary neurons.

The present disclosure of KP acting as an analgesic in a bone cancer pain model is novel and noteworthy, particularly in the arena of cancer pain. This finding requires additional research to further elucidate the underlying cellular and neural circuit mechanisms, e.g. in the spinal cord dorsal horn (SCDH). Interestingly, effective analgesia was observed at the higher KP dose whereas bone lesions caused by implanted cancer cells were not significantly affected. Importantly, higher doses of KP can be used to address whether there are anti-proliferative effects of the compound. This question can also be asked when using KP in combination with a lower dose of anti-growth compound that by itself would not affect tumor cells' proliferation. Given the established roles of CDKs and GSK3 in growth-regulation of cancer cells, studies were done to determine whether KP could enhance co-applied cell-ablative strategies, and whether higher doses of KP might improve analgesia and contain cancer cell growth. Also, the anti-proliferative effects of targeted overexpression of δ-catenin by the tumor cells were investigated whether it had an anti-proliferative effect.

The approach as described here for pain targeting is innovative. The enhanced Kcc2/KCC2 gene expression, based on kenpaullone (KP) treatment as presented here, complements direct enhancement of KCC2 chloride extrusion in targeting pathologic pain. Complementary use will help overcome recalcitrant lack of expression and function of KCC2 in pain relay neurons, as might be expected in clinical cases of “refractory” chronic pain. Clinical combination-use of KCC2 expression enhancers with analgesic compounds that have different mechanisms of action will be advantageous because renormalized inhibitory transmission will likely cause improved effectiveness of other compounds, such as gabapentinoids.

The following Examples are provided by way of illustration and not by way of limitation.

### EXAMPLES

### Materials and Methods

Screening in Primary Cortical Neurons from Kcc2-LUC Transgenic Mice

Transgenic mice expressing red-shifted luciferase (LUC) under the control of the Kcc2 promoter (−2052/+476) (Yeo et al., 2009, J Neurosci 29: 14652-14662) inserted into the Rosa26 locus were previously described (Liedtke et al., 2013, Small (Weinheim an der Bergstrasse, Germany) 9: 1066-1075; Yeo et al., 2013, Proc Natl Acad Sci USA 110:4315 4320). Primary cortical neuronal cultures were generated from newborn (p0) mice of this line.

Cytosine arabinoside (2.5 μm) was added to cultures on the second day after seeding [2 d in vitro (DIV)] to inhibit the proliferation of non-neuronal cells. Cell suspension was plated at a density of 1×10⁶ cells/ml onto 24-well tissue-culture dishes coated with poly-d-lysine. Cortical neuronal cultures prepared by this method yielded a majority population of neuronal cells, with negligible glia contamination, as evidenced by the absence of GFAP by Western blotting.

After a week in culture, neurons were treated with compounds (100 nM for 48 h). LUC activity was then determined for each compound. Culture supernatant was removed and cells were lysed with 150 μl lysis buffer (Targeting Systems CA, USA cat. CLR1). LUC activity was measured with a RedLuciferase Assay kit (Targeting Systems cat. FLAR) according to the manufacturer's instructions. A Veritas microplate luminometer was used to measure luminescence; for each treatment triplicates of 40 μl cell lysates (from each 24-well tissue-culture dish) were used and 25 μl substrate was injected per well. To evaluate the quality of screening methodology, Z′ factor (Zhang et al., 1999, Journal of biomolecular screening, 4: 67-73) was ascertained using 0.5% (v/v) DMSO as negative control and Trichostatin A as positive control. Relative Light units (RLU) for each treatment was derived from Light Units (compound treatment)/vehicle treatment (0.5% DMSO).

The primary screen encompassed three levels of LUC measurements. The first level of screening yielded a total of 137 compounds with cut-off RLU>125% LUC activity. These 137 compounds were subject to second round of screening, which was carried out in duplicate independent assays to yield the top 103 compounds sorted for highest RLU. The 103 compounds were then subjected to a third round of screening carried out in duplicate independent assays to yield the top 40 compounds which were again ranked. The best 22 of these 40 compounds (ranked for highest RLU activity) were subjected to secondary screening: cultured neurons were treated with the 22 compounds; RT-qPCR and Clomeleon imaging methodologies (Yeo et al., 2009, J Neurosci 29: 14652-14662) were used to determine effects of compounds on Kcc2 mRNA expression and [Cl-]i, respectively. Each compound was ranked based on composite scores from primary and secondary screening.

### Human Neuronal Cultures

The neuron-enriched cultures were established from fetal cortical specimens at 15-20 weeks of gestation. The protocols for tissue processing complied with all federal and institutional guidelines. The cultures were plated on PEI (polyethyleneimine solution) substrate and maintained in Neurobasal media supplemented with B27 as described (Pelsman et al., 2003, International Journal of Developmental Neuroscience 21: 117-124; Yeo et al., 2013, Proc Natl Acad Sci USA 110: 4315-4320).

For RT-qPCR, the cell pellets were collected after treatments with vehicle (0.1% DMSO) or KP (50, 100, and 400 nM) for 48 h starting at Day 6 in vitro.

For immunostaining, the cultures were treated for 2-4 days with vehicle or 400 nM KP, fixed with 4% PFA at Day 10, and processed for double-staining with anti-KCC2 and anti-synaptophysin or anti-NeuN (Table 1).

TABLE 1
Primary Antibodies

| Antibody | Source |
|---|---|
| anti-KCC2 | Millipore (07-432) (mouse spinal cord) |
| | ThermoFisher Scientific (PA5-78544) |
| | (human, mouse, rat ICC) |
| anti-β_{III} Tubulin | Abcam (mouse, ab78078; rabbit, ab229590) |
| anti-β-catenin | SigmaAldrich (PLA0230) |
| anti-δ-catenin | SigmaAldrich (MABN2254) |
| anti-Synaptophysin | ThermoFisher (MA1-213) |
| anti-FLAG | SigmaAldrich (F3165) |
| anti-NeuN | BioLegend (834501) |

The 0.34 micrometer confocal slices through entire cell layers at different optical fields (n=17-28 for each group) of the fixated cultures were acquired using a Zeiss LSM700 confocal microscope.

Image Z-stacks were analyzed using Imaris 9.2.1 software. Optical density intensity sum values for each stack/channel were divided by corresponding data volumes and the resulting values were normalized to the number of specifically labeled cells within each stack. There were no significant differences in the number of cells between vehicle and KP-treated groups.

### Kcc2 Promoter Luciferase Reporter Assays

A fragment of the mouse Kcc2 gene promoter (position −2052 kbp to +476 kbp) was amplified from genomic DNA prepared from cultured mouse primary glial cells. A 2.5 kb PCR fragment was cloned into the pGL4.17-Basic Vector (Promega) to generate the Kcc2 promoter reporter construct. TCF and Kaiso binding sites were identified in this fragment. Using wild-type construct pGL4.17 Kcc2 as a template, site-directed mutagenesis using Phusion DNA Polymerase enzyme (Thermo Fisher F549L) in conjunction with complementary primers bearing the specific mutation were used to mutate the Kaiso and TCF DNA-binding sites. PCR was followed by Dpnl enzyme digestion to remove parental plasmid DNA. All constructs were verified by sequencing.

N2a cells were grown to 90% confluency in 24-well dishes in 0.4 mL of medium (DMEM, 2% Fetal Bovine Serum, 2 mM glutamine, 1% Non-essential amino acids, and 1% Penicillin/Streptomycin). Cells were transiently transfected using TurboFect reagent (Thermo Fisher R0531), with 500 ng of the pGL4.17-constructs plus 20 ng of the control Renilla plasmid (Promega, E2231) to normalize for transfection efficiency. Twenty-four hours after transfection, luminescence was measured using the Dual-Luciferase® Reporter Assay System (Promega) in a microplate luminometer (Veritas, Turner Biosystems). Mutant promoters were compared to WT, and the response of the respective promoter to KP (400 nM) was measured. Three independent transfection experiments were carried out and LUC assays were done in triplicates for each transfection. RLU is expressed as firefly luciferase activity relative to Renilla LUC activity.

### RT-qPCR

OligodT-initiated reverse transcription of 1 μg total RNA, DNA-se treated, was subjected to RT-qPCR using primers specific for Kcc2 for rat and KCC2 for human sequences, normalized for neuronal β_{III}-tubulin, as previously described (Yeo, et al., 2009, J Neurosci 29: 14652-14662). For cultured neurons, total RNA was extracted from pelleted cells, and for spinal cord tissue it was extracted from microdissected lumbar spinal cord dorsal horn.

Total RNA was isolated from cultured cell samples using Directzol RNA miniprep kit (ZymoResearch). The protocol includes DNAse digestion to exclude genomic DNA from preparations. Total RNA (1 μg) was reverse transcribed using oligo primers (dT) and SuperScriptIII first-strand synthesis kit (Invitrogen). Gene expression was assessed by quantitative real-time PCR using 2×SYBR Green Master Mix (Qiagen) and a three-step cycling protocol (anneal at 60° C./elongate at 72° C., denature at 95° C.). Specificity of primers was verified by dissociation/melting curve for the amplicons when using SYBR Green as a detector. All reactions were performed in triplicates. The amount of target messenger RNA (mRNA) in the experimental group relative to that in the control was determined from the resulting fluorescence and threshold values (Ct) using the AACt method. βiirtubulin was used as a housekeeping gene.

### Behavioral Assessments

For pain-related behavior, mechanical allodynia was assessed with von Frey filaments (Ugo Basile, Italy). Animals were habituated to the testing environment daily for at least 2 days before baseline testing. The room temperature and humidity remained stable for all experiments. The mice were placed on a 5×5-mm wire-mesh grid floor in individual compartments to avoid visual stimulation and allowed to adapt for 0.5 h prior to the von Frey test. The von Frey filament was then applied to the middle of the plantar surface of the hind paw, perpendicularly, with a series of von Frey hairs with logarithmically increasing stiffness (0.02-2.56 g, Stoelting). The withdrawal responses following the hind paw stimulation were measured at least three times. The 50% paw withdrawal threshold was determined by up-down method (Chen et al., 2015, J Clin Invest 125: 3226-3240).

For assessment in rotarod (RR), all animals received training prior to experiments; mice were placed on the RR apparatus set in an accelerating rotational speed mode (3-30 rpm, 300 s max/trial). Following training, the average time to fall from the rotating cylinder over three trials was recorded as baseline latency (4-40 rpm, 300 s max/trial). Mice were injected daily with either vehicle or drug compounds before RR tests. Latency to fall was measured (4-40 rpm, 300 s max/trial (inter-trial interval is at least 15 min). The average latency to fall from the rod was recorded for each animal.

Conditioned place preference (CPP) was conducted using a CPP box, which consists of two conditioning chambers distinguished by visual and sensory cues, along with a small buffering chamber. All mice received a 3-day preconditioning habituation period with free access to both conditioning chambers and the time spent in each chamber was recorded for 15 min on Day 3 after habituation. On conditioning days (Day 4-Day 10), mice first received the vehicle control intraperitoneally (i.p.) (5% DMSO, 5% Tween-80 in normal saline) paired with a randomly chosen chamber in the morning. After 4 hours, mice received KP (i.p. 30 mg/kg) or vehicle, paired with the other chamber. During the conditioning, mice were allowed to stay only in the paired chamber for 15 min without access to other chambers. On test day (dll), mice were placed in the buffering chamber with free access to both conditioning chambers and choice behavior was recorded for 15 min. The CPP scores were calculated as post-conditioning time minus preconditioning time spent in the paired chamber.

### Bone Cancer Pain Model and Bone Imaging

Murine lung carcinoma cell line LLC1 (ATCC CRL-1642) was digested with 0.25% trypsin and suspended at 5×10⁷/ml cells in PBS. Following previous protocol (Honore et al., 2000, Nat Med 6: 521-528), mice were anesthetized with 3% isoflurane (oxygen flow: 1.0 L/min). The left leg was shaved, and the skin was disinfected with 10% povidone-iodine and 75% ethanol. A 0.5-1 cm superficial incision was made near the knee joint to expose the patellar ligament. Then a 25-gauge needle was inserted at the site of the intercondylar notch of the left femur into the femoral cavity and the needle was then replaced with a 10 μl microinjection syringe containing 4 μl suspension of tumor cells (2×10⁵) and 2 μl absorbable gelatin sponge solution for the inside closure of the injection site. The contents of the syringe were slowly injected into the femoral cavity (2 min). To further prevent leakage of tumor cells from the bone's cavity, the outside injection site was sealed with silicone adhesive at the periost level (Kwik-Sil, World Precision Instrument, US). Animals with surgery related movement dysfunction or with mis-targeted tumor cell injection (i.e., injection that occurred outside bone tumor) were excluded.

Osteolytic bone destruction was continuously assessed through radiography using a Faxitron system (Faxitron Bioptics LLC, Tucson, Ariz.). Radiographs of tumor-bearing femora were rated following a 0-5 score scale as previously described (Yang et al., 2015, J Neurosci 35: 7950-7963; Honore et al., 2000, Nat Med 6: 521-528): 0—normal bone without signs of destruction; 1—one to three radiolucent lesions indicative of bone destruction; 2—increased number of lesions (three to six lesions) and loss of medullary bone; 3—loss of medullary bone and erosion of cortical bone; 4—full-thickness unicortical bone loss; 5—full-thickness bicortical bone loss and displaced skeletal fracture. All radiographic image quantifications were obtained in a blinded fashion.

### Chromatin Immunoprecipitation (ChIP)

ChIP assay was carried out as described previously (Yeo et al., 2009, J Neurosci 29: 14652-14662; Yeo et al., 2013, Proc Natl Acad Sci USA 110: 4315-4320). Primary cortical neurons (0.7×10⁶) were used for each ChIP experiment. Cells were crosslinked with 1% formaldehyde for 30 min, washed twice with cold PBS, resuspended in lysis buffer [1% SDS, 10 mm EDTA, and 50 mm Tris-HCl, pH 8.0, with protease inhibitor cocktail (Roche)], and sonicated for 15 s pulses. The lysates were clarified by centrifugation at 10,000 rpm for 10 min at 4° C. in a microcentrifuge. One-tenth of the total lysate was used as input control of genomic DNA. Supernatants were collected and diluted in buffer (1% Triton X-100, 2 mm EDTA, 150 mm NaCl, 20 mm Tris-HCl, pH 8.0, and protease inhibitor cocktail) followed by immunoclearing with 1 mg of salmon sperm DNA, 10 ml of rabbit IgG, and 20 ml of protein A/G-Sepharose (Santa Cruz Biotechnology) for 1 h at 4° C. Immunoprecipitation was performed overnight at 4° C. with 2 mg of each specific antibody. Precipitates were washed sequentially for 10 min each in TSE1 buffer (0.1% SDS, 1% Triton X-100, 2 mm EDTA, 150 mm NaCl, and 20 mm Tris-HCl, pH 8.0), TSE2 (TSE1 with 500 mm NaCl), and TSE3 (0.25 m LiCl, 1% NP-40, 1% deoxycholate, 1 mm EDTA, and 10 mm Tris-HCl, pH 8.0). Precipitates were then washed twice with 10 mm Tris/0.1 mm EDTA, pH 7.8 and extracted with 1% SDS containing 0.1 m NaHCO₃. Eluates were pooled and heated at 65° C. for 4 h to reverse formaldehyde crosslinking. DNA fragments were purified with Qiagen Qiaquick kit. For ChIP PCR, 1 μl of a 25 μl DNA extraction was used.

### Immuno-Cytochemistry of Cultured Neurons

Immunocytochemistry labeling of cultured neuronal cells was carried out as previously described (Liedtke et al., 2013, Small (Weinheim an der Bergstrasse, Germany) 9: 1066-1075; Yeo et al., 2009, J Neurosci 29: 14652-14662; Yeo et al., 2013, Proc Natl Acad Sci USA 110: 4315-4320). Primary antibodies are shown in the antibody table (Table 1). Anti-KCC2 primary antibodies were validated with developing rat primary cortical neurons; an increase in staining pattern was observed that tightly matched increase of Kcc2 mRNA expression as previously described (Liedtke et al., 2013, Small (Weinheim an der Bergstrasse, Germany) 9: 1066-1075; Yeo et al., 2009, J Neurosci 29: 14652-14662; Yeo et al., 2013, Proc Natl Acad Sci USA 110: 4315-4320). Secondary antibodies used were goat anti-mouse IgG Alexa Fluor 594 (Invitrogen A11032) and goat anti-rabbit IgG Alexa Fluor 594 (Invitrogen A11012). DAPI stain was obtained from Sigma Aldrich (D9542). Stained cells were observed using an inverted confocal microscope (Zeiss LSM780).

Stacks of images recorded at 0.35 μm intervals through separate channels with a 63× oil-immersion lens (NA, 1.40, refraction index, 1.45) were obtained. Zen software (Zeiss) was used to construct composite images from each optical series by combining the images recorded through the different channels, and the same software was used to obtain Z projection images (image resolution: 1024×1024 pixels; pixel size: 0.11 μm). ImageJ was used for morphometry.

### Spinal Cord Immuno-Histochemistry

All mice were deeply anaesthetized with isoflurane and then transcardially perfused with ice-cold 4% paraformaldehyde in 0.1 M phosphate buffer, pH 7.4 (4% PFA). Dissected spinal cord samples were then post-fixed overnight in 4% PFA at 4° C., cryoprotected in a 20% sucrose solution in PBS at 4° C., frozen in Tissue-Tek OCT (Sakura), and stored at −80° C. until sectioning. Samples were sectioned at 20 μm using a cryostat (Microm HM 505N). The sections were blocked with 2% bovine serum albumin (BSA) in PBS with 0.3% Triton X-100 (Blocking solution) at room temperature for 1 h. The sections were treated with primary antibody in blocking solution at 4° C. overnight. The sections were washed three times followed by secondary antibody treatment at 4° C. for 2 hours. Anti-KCC2 antibody was validated as described above for immuno-cytochemistry. The goat anti-rabbit IgG Alexa Fluor 488 was obtained from Invitrogen (A-11008). Morphometry was conducted using ImageJ with region-of-interest Rexed laminae I-II.

### Chloride Imaging

Chloride imaging of primary cultured cortical neurons was conducted as previously described (Kuner and Augustine, 2000, Neuron 27: 447-459; Liedtke et al., 2013, Small (Weinheim an der Bergstrasse, Germany) 9: 1066-1075; Yeo et al., 2009, J Neurosci 29: 14652-14662). A Clomeleon expression plasmid was transfected into primary cortical neurons by electroporation (Amaxa Nucleofector Device). Transfected neurons were verified by yellow fluorescent protein (YFP) fluorescence, and ratiometric images (excitation at λ=434 nm, dual emission at λ=485 and 535 nm; for resting chloride, six stable frames at a rate 12 of per minute were captured, which were averaged) were acquired using RATIOTOOL program. Calibration of Clomeleon signals (535 nm/485 nm emission ratio) was performed by using tributyltin-nigericin to establish a standard curve, which was then normalized for measured intraneuronal pH to take into account the pH sensitivity of Clomeleon.

### Spinal Cord Dorsal Horn Electrophysiology

For spinal cord slice preparation, adult (5-7 weeks) male mice were anesthetized with urethane (1.5-2.0 g/kg, i.p.). The lumbosacral spinal cord was microsurgically removed and submerged into ice-cold dissection media which was saturated with 95% O₂ and 5% CO₂ at room temperature. After extraction and still under anesthesia, animals were euthanized. Transverse slices (300-400 μm) were cut using a vibrating microslicer (VT1200 s Leica). The slices were incubated at 32° C. for at least 30 min in regular artificial cerebrospinal fluid (aCSF), equilibrated with 95% O₂ and 5% CO₂.

The following solutions were used: Dissection solution: Sucrose 240 mM, NaHCO₃25 mM, KCl 2.5 mM, NaH₂PO₄ 1.25 mM, CaCl₂ 0.5 mM, MgCl₂ 3.5 mM (Cheng et al., 2017, Nature neuroscience 20: 804-814). Regular artificial cerebrospinal fluid (ACSF): NaCl 117 mM, KCl 3.6 mM, MgCl₂ 1.2 mM, CaCl₂ 2.5 mM, NaHCO₃25 mM, NaH₂PO₄ 1.2 mM, glucose 11 mM. The pH value of ACSF or dissection solution was adjusted to 7.4 when saturated with the gas. Normal intrapipette solution (pH 7.2 and 310 mOsm): K-methylsulfate 115 mM, KCl 25 mM, MgCl₂ 2 mM, HEPES 10 mM, GTP-Na 0.4 mM and Mg-ATP 5 mM.

Electrophysiological recordings were conducted as follows. A slice was placed in the recording chamber and completely submerged and superfused at a rate of 2-4 ml/min with aCSF saturated with 95% O₂ and 5% CO₂ at room temperature. Perforated patch-clamp was used to avoid alteration of the [Cl-]i. To measure the chloride equilibrium potential (Eci), gramicidin D (80 μg/mL with 0.8% DMSO final concentration, from an 8 mg/mL stock in DMSO) was added to the intrapipette solution, and 6-cyano-7-nitroquinoxaline-2,3-dione (CNQX, 10 μM), DL-2-amino-5-phosphonovaleric acid (APV, 50 μM), and tetrodotoxin (TTX, 0.5 μM) were added to the aCSF solution. The tip of the patch pipette was filled with the normal intrapipette solution while the rest of the pipette contained the gramicidin-containing solution. After forming a seal on the membrane, ˜30 min was given for the gramicidin to induce sufficient cation-selective pores in the membrane and lowered the series resistance to below 100 MS2. Membrane potential measurements were corrected for liquid junction potential, which was measured as previously described (Jiang et al., 2014, Journal of neurophysiology 111: 991-1007). GABA (1 mM) was puffed locally and instantaneously, and the puff pipette was aimed toward the recording pipette. To determine the reversal potential of GABA-evoked currents, voltage ramps were applied from +8 to −92 mV over 200 ms at a holding potential of −42 mV. Since the voltage ramp might elicit a basal current, a control voltage ramp was applied, and 1 min later GABA was puffed followed by another voltage ramp (Billups and Attwell, 2002, The Journal of physiology 545: 183-198). The reversal potential was analyzed.

Signals were acquired using an Axopatch 700B amplifier and analyzed with pCLAMP 10.3 software. Only neurons with resting membrane potential <−50 mV and stable access resistance were included.

### Drug Affinity Responsive Target Stability (DARTS) Assay

DARTS assay was conducted using 20 μM KP as “bait” and LC-MS/LC to identify proteins bound to KP as previously described (Lomenick, et al., 2009, Proc Natl Acad Sci USA 106: 21984-21989). The assay was conducted in both the presence and absence of detergents NP40 and N-dodecyl-b-D-maltoside.

In detail, cultured primary rat cortical neurons were treated with either vehicle DMSO (0.1%) or 20 μM KP for 30 h. Cells were lysed in ice-cold lysis buffer (Tris.Cl pH8 50 mM, NaCl 150 mM, NP40 0.5%, N-dodecyl-b-D-maltoside 0.5%, Phosphatase Inhibitor (Pierce #88667) and Protease Inhibitor (Roche #11836153001)). Protein concentrations were determined by Bio-Rad DC Protein Assay kit using bovine albumin as standard. All steps were performed on ice. Samples were warmed to room temperature and digested with pronase (final concentration 1:500) for 30 min at 30° C. Digestion was halted using 0.5 M EDTA. Only proteins not bound to KP were digested. The protein mixture was dialyzed using dialysis cassettes (Thermo Fisher 66203, 2K MWCO) and analyzed by LC-MS/MS method to identify proteins that are bound to KP, the latter step carried out in the Duke Proteomics Core Laboratory.

### Kinome Analysis

Cultured rat primary cortical neurons were treated with either vehicle DMSO (0.1%) or 1 μM KP for 1 h/24 h. Cells were lysed in non-detergent-containing buffer (Tris.Cl pH8 50 mM, NaCl150 mM, 0.5% Phosphatase Inhibitor (Pierce #88667) and Protease Inhibitor (Roche #11836153001)). 500 μl was removed and solid urea was added to a final concentration of 8 M. Samples were sonicated for further solubilization. After clearing of insoluble material by centrifugation, protein concentration was measured by Bradford assay. 250 μg of total protein was removed from each sample and solubilization buffer was added to normalize all samples to 0.93 μg/μl protein. Samples were then spiked with bovine alpha-casein to 30 fmol/μg of total protein. Samples were reduced with 10 mM DTT at 32° C. for 45 min and then alkylated with 20 mM iodoacetamide at room temperature for 30 min. Samples were trypsin digested at 1:25 (enzyme-to-protein) overnight at 32° C. Following acidification with TFA to pH 2.5, samples were subjected to a C18 solid-phase extraction cleanup. Eluted peptides were split 80% for phosphopeptide analysis and 20% reserved for unbiased differential expression. The phosphopeptide fraction (200 μg) was then frozen and lyophilized prior to phosphopeptide enrichment.

TiO2 Enrichment: Samples were re-suspended in 65 μl of 1M glycolic acid in 80% MeCN/1% TFA and were enriched on TiO2 resin using a 10 μl GL Sciences microliter TiO2 spin tips. After elution and acidification, samples were lyophilized to dryness and resuspended in 100 μl of 0.15% TFA in water. After cleanup using a C18 STAGE tip, and resuspension in 2% acetonitrile, 0.1% TFA, 10 mM citric acid samples were quantified.

Quantitative analysis of Phosphopeptide Enriched Samples: Quantitative LC-MS/MS was performed in singlicate (4 uL=33% of the total sample each injection) for phosphopeptide-enriched samples using a nanoAcquity UPLC system (Waters Corp) coupled to a Thermo QExactive Plus high resolution accurate mass tandem mass spectrometer (Thermo) via a nanoelectrospray ionization source. Briefly, the sample was first trapped on a Symmetry C18 300 mm Å˜180 mm trapping column for 6 min at 51/min (99.9/0.1 v/v water/acetonitrile 0.1% formic acid), after which the analytical separation was performed on a 1.7 μm Acquity BEH130 C18 75 mm Å˜250 mm column (Waters Corp). Peptides were held at 3% acetonitrile with 0.1% formic acid for 5 min and then subjected to a linear gradient from 3 to 30% acetonitrile with 0.1% formic acid over 90 min at a flow rate of 400 nL/min at 55° C. Data collection on the QExactivePlus mass-spec was performed in a data-dependent acquisition (DDA) mode following protocol of the manufacturer.

### Statistics

All data are expressed as mean±SEM. Differences between groups were evaluated using two-tailed Student's t test (experimental against sham control), or in the case of multiple groups, one-way ANOVA followed by post-hoc Bonferroni test. The criterion for statistical significance is p<0.05.

### Example 1: Identification of Kcc2 Gene Expression Enhancers

To identify Kcc2 gene expression enhancing compounds, primary cortical neurons from Kcc2- luciferase (LUC)-knockin (Kcc2-LUCki) mice (Liedtke, et al., 2013, Small 9: 1066-1075; Yeo, et al., 2013, Proc Natl Acad Sci USA 110: 4315-4320) were cultured and LUC was used as readout for activity of the proximal Kcc2 promoter (2.5 kB), which drives LUC in this transgenic mouse line. A Z′ factor of 0.94 was recorded in the screening assay. It was recognized that such strategy would not select for long-range enhancers of Kcc2 gene expression that act outside the 2.5 kB core Kcc2 promoter.

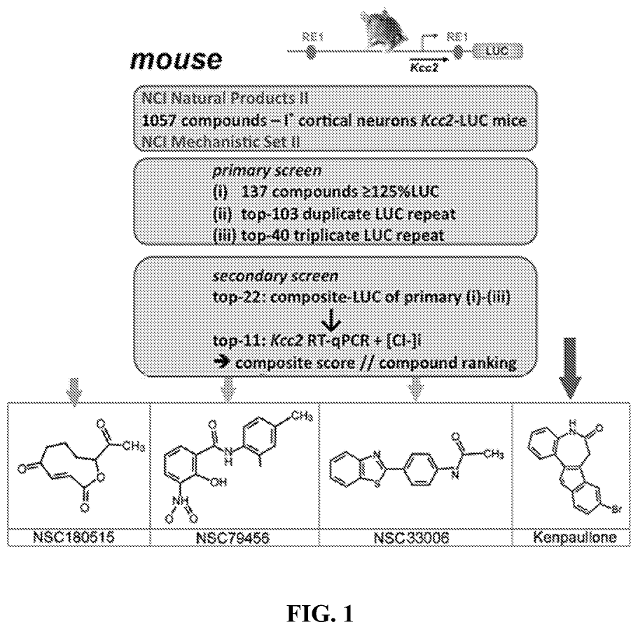

1057 compounds were screened and contained in two NCI libraries: NCI compound libraries Natural Products II and Mechanistic Diversity Set II (FIG. 1, Table 2), related to inhibition of growth of malignantly transformed cells. As shown in FIG. 1, three rounds of primary screen based on luciferase (LUC) activity were conducted in primary mouse neurons and followed by secondary screen including Kcc2 RT-qPCR and Clomeleon chloride imaging. Iterative screening followed by measurements of Kcc2 mRNA (RT-qPCR) and intracellular chloride ([Cl-]i) (clomeleon chloride indicator protein) led to decreasing numbers of re confirmed hits, which eventually identified four (4) “winner” compounds listed in the bottom panel of FIG. 1.

TABLE 2
Iterative Screening Compounds

| Luciferase | RT-qPCR | Clomeleon | | | Chloride | Chloride | |
|---|---|---|---|---|---|---|---|
| Score | Score | Score | Total | Top 2 | [Cl—]i | reduction | |
| (triplicates) | (triplicates) | (duplicates) | Score | Hits | mM | (%) | Natural Compounds |
| 1 | 5 | 5 | 11 | — | 75 => 42 | 56 | confertifoline |
| 2 | 2 | 2 | 6 | winner | 68 => 23 | 33.8 | kenpaullone |
| | | | | | | | (9-bromo-7,12-dihydro-indolo |
| | | | | | | | [3,2-d] [1]benzazepin-6(5H)- |
| | | | | | | | one) |
| 3 | 4 | 6 | 13 | — | 75 => 43 | 57.3 | anisomycine |
| 4 | 1 | 1 | 6 | winner | 68 => 22 | 32 | NSC180515 |
| | | | | | | | [2-Acetyl-2,3,4,5- |
| | | | | | | | tetrahydrooxonine-6,9-dione] |
| 5 | 3 | 4 | 12 | — | 75 => 39 | 52 | tylocrebrine |
| 6 | 6 | 3 | 15 | — | 68 => 33 | 48.5 | cantharidine |
| 7 | — | — | — | — | — | — | veratridine |
| 8 | — | — | — | — | — | — | |
| 9 | — | — | — | — | — | | proresidor |
| 9 | — | — | — | — | — | — | ketoconazole |
| 10 | — | — | — | — | — | — | colchicine |
| 1 | 3 | 1 | 5 | winner | 74 => 24 | 32.4 | NSC79456 |
| | | | | | | | [n-(2,4-Dimethylphenyl)-2- |
| | | | | | | | hydroxy-3-nitrobenzamide] |
| 2 | 4 | 5 | 11 | — | 82 => 40 | 48.8 | 9-Deazaadenosine |
| 2 | 1 | 2 | 5 | winner | 74 => 28 | 37.8 | NSC33006 |
| | | | | | | | [N-[4-(1,3-benzothiazol-2- |
| | | | | | | | yl)phenyl]acetamide] |
| 3 | 2 | 3 | 8 | — | 74 => 32 | 43.2 | 4-Ipomeanol |
| 4 | 5 | 4 | 13 | — | 82 => 38 | 46.3 | 4-(1,3-Benzothiazol-2-yl)-2- |
| | | | | | | | methylaniline |
| 5 | — | — | — | — | — | — | Quinolinium, dichloride |
| 5 | — | — | — | — | — | — | (2E)-N-(2-methylphenyl) -2- |
| | | | | | | | [(3-nitrophenyl)methylidene]- |
| | | | | | | | 3-oxobutanamide |
| 5 | — | — | — | — | — | — | Akt Inhibitor V |
| 6 | — | — | — | — | — | — | Sauzivamycin |
| 6 | — | — | — | — | — | — | 3,3,4,4-tetramethyloxolane- |
| | | | | | | | 2,5-diol |

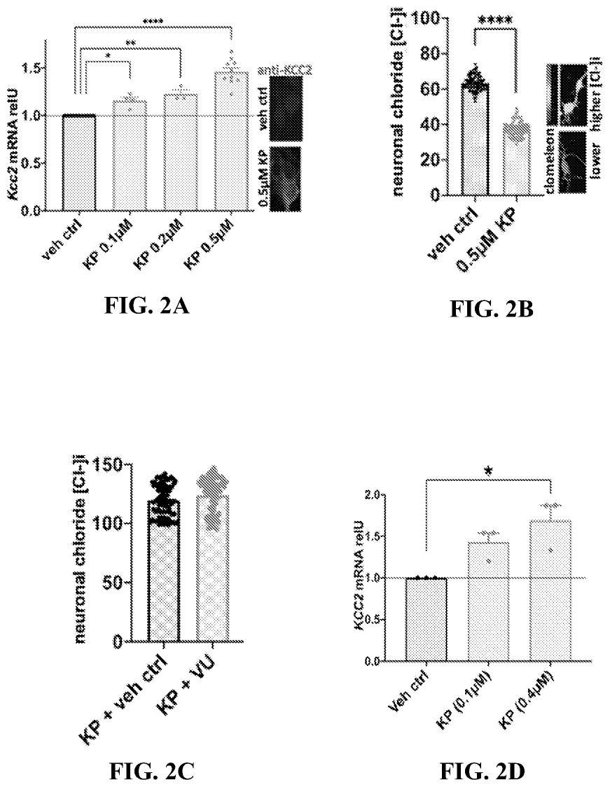

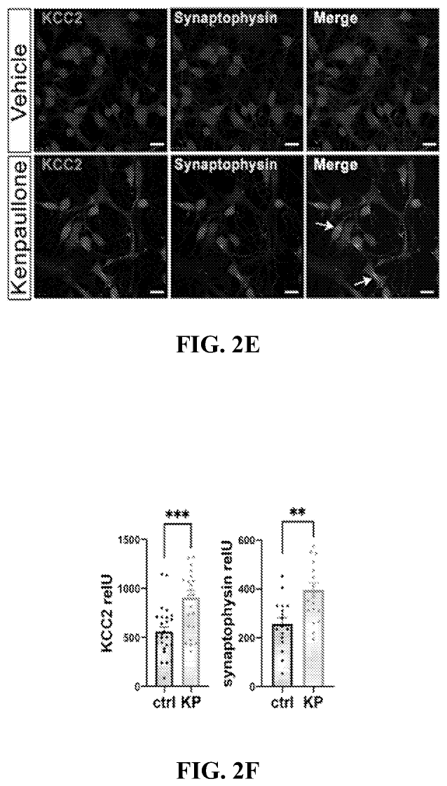

Among the compounds that were screened, kenpaullone (KP) (9-bromo-7,12-dihydro-indolo [3,2-d] [1]benzazepin-6(5H)-one), a GSK3/CDK kinase inhibitor, was identified as a promising compound for further study based on its previous record of neuroprotection in translationally relevant preclinical models (Liu et al., 2016, Cell Reports 14: 115-128; Reinhardt et al., 2019, Stem Cell Reports 12: 502-517; Skardelly et al., 2011, Neuroscience 29: 543-547; Yang et al., 2013, Cell Stem Cell 12: 713-726). The data from the further study establish that KP enhanced Kcc2 gene expression and function in rat and human primary cortical neurons (FIGS. 2A-2F).

Specifically, Kcc2 mRNA expression in primary rat cortical neurons was increased in a dose-dependent manner after KP treatment, which was also reflected by increased protein expression as shown by KCC2 immuno-label (micrographs) (FIG. 2A). Results represent the average mRNA expression of multiple independent neuronal cultures [n=12 (control), n=4 (0.1 μM KP), n=3 (0.2 μM KP), n=10 (0.5 μM KP). *p<0.05, **p<0.01, ****p<0.0001, one-way ANOVA]. Additionally, rat neuronal [Cl-]i, measured with ratiometric chloride indicator, clomeleon, was robustly and significantly reduced after KP treatment (n≥75 neurons/3 independent cultures; ****p<0.0001, t-test) (FIG. 2B). It was observed that add-on treatment with KCC2-transport blocker, VU0240551 (2.5 μM), led to a [Cl-]i≥120 mM, for both vehicle-treated and KP-treated rat neurons, indicating that KP's chloride lowering effect relies on KCC2 chloride extruding transport function (FIG. 2C; n≥75 neurons/3 independent cultures).

Similarly, Kcc2 mRNA expression in primary human cortical neurons was increased in a dose-dependent manner upon treatment with KP (FIG. 2D). Results represent the average mRNA expression of 3 independent neuronal cultures (*p<0.05, one-way ANOVA). This finding was accompanied by increased protein expression of KCC2 and synaptophysin (FIGS. 2E-2F), both of them co-localizing. Synaptophysin was used as a marker of synaptic maturation and generally of a mature neuronal phenotype, which was rooted in increased expression of KCC2. Representative confocal images at DIV 10 immuno-labelled for KCC2 and synaptophysin after vehicle or KP-treatment were in shown in FIG. 2E. Co-localization was noted by yellow arrow therein (Bar, 10 μm). Morphometry of immuno-cytochemistry shows 62% increased KCC2 (FIG. 2F, left hand) and 54% increased synaptophysin (FIG. 2F, right-hand) expression after KP treatment, relative to vehicle treatment/control (n=27-28 neurons analyzed for KCC2, ***p<0.001; n=17-18 neurons analyzed for synaptophysin, *p<0.01, t-test).

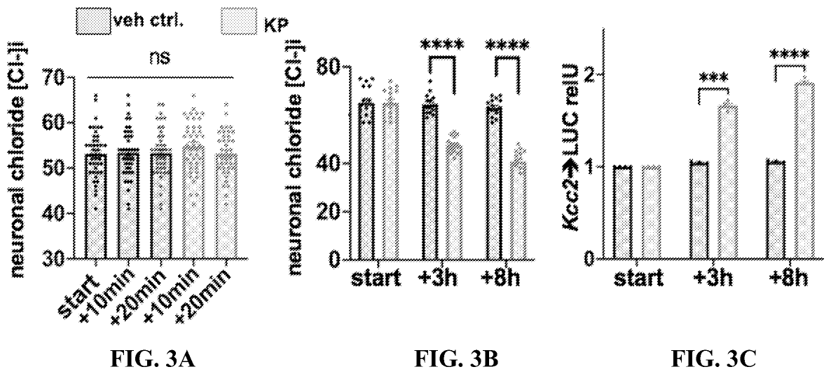

The data further show that KP did not function as an enhancer of KCC2 transporter-mediated chloride efflux, although it affected Kcc2 expression and function within 3 h of treatment in rat primary cortical neurons (FIGS. 3A-3C). Specifically, no difference was observed in neuronal chloride levels, measured with clomeleon fluorescent indicator after KP treatment vs. vehicle treatment at the 10 min and 20 min time-points (FIG. 3A). Clomeleon-based measurement of [Cl-]i was significant decreased at the 3 h time-point, even more pronounced at 8 h time-point (FIG. 3B; n=16 neurons/group, ****p<0.001, 2-way ANOVA). When rat primary cortical neurons were transfected with renilla luciferase and driven by Kcc2 promoter, significantly increased activation of the Kcc2 promoter was observed at 3 h time-point, more pronounced at 8 h time-point (FIG. 3C; ***p<0.001, ****p<0.0001, 2-way ANOVA). It is shown that KP enhanced Kcc2 gene expression and KCC2 chloride extrusion function with rather rapid kinetics within 3 h time frame.

These findings in rodent and human neurons indicate that KP enhanced Kcc2/KCC2 gene expression but not KCC2-mediated chloride extrusion in CNS neurons. That is, KP functioned as Kcc2/KCC2 gene expression enhancer in mammals including humans. Enhanced synaptic maturation and increased KCC2 expression were uncovered in human primary cortical neurons, although most likely causally linked.

### Example 2: Function of Kenpaullone as an Analgesic In-Vivo

In-vivo analgesic effects of systemically applied kenpaullone (KP) were evaluated. To do so, two types of preclinical mouse models of pathologic pain were used: (1) peripheral nerve constriction-induced neuropathic pain, using the PSNL method of nerve constriction injury, and (2) a bone cancer pain model that relies on implantation of mouse lung carcinoma cells into the marrow of the femur. Nerve constriction injury, a widely used neuropathic pain model, was previously used to demonstrate down-regulated KCC2 expression in the spinal cord dorsal horn in pain. Bone cancer pain is another clinically-relevant pain model. In particular, it fits the profile of KP, as the compound inhibits GSK3β and CDKs, which might possibly be analgesic but also important for growth regulation of the implanted carcinoma cells.

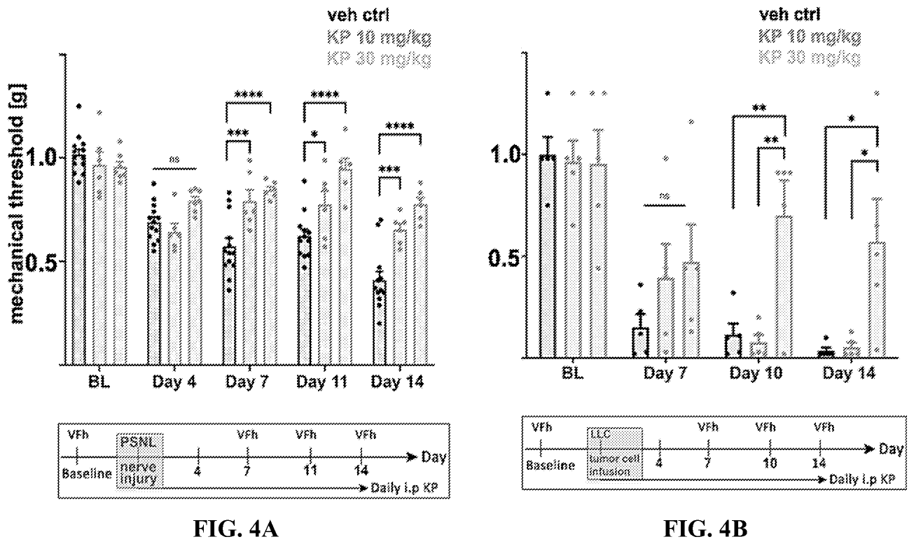

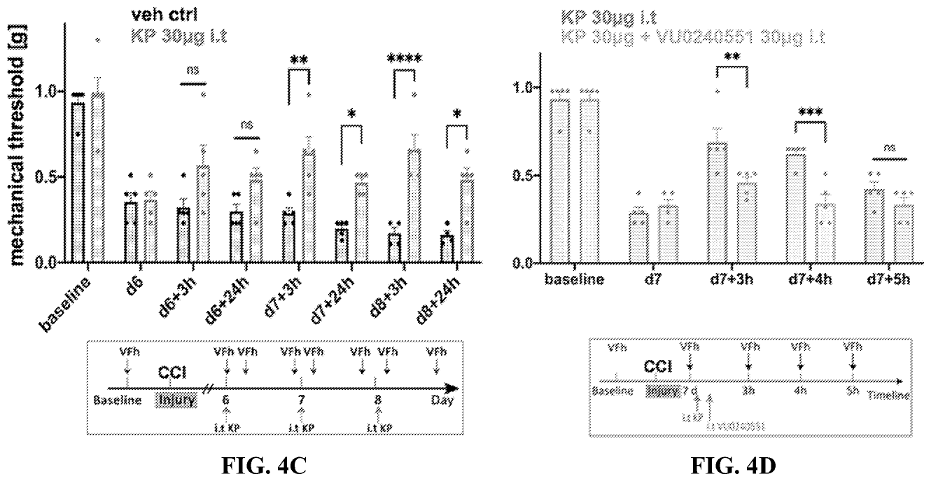

It was found that behavioral sensitization (i.e., mechanical allodynia) in both pain models, namely decrease in withdrawal thresholds in response to mechanical cues, was significantly reduced by KP treatment, as illustrated in FIGS. 4A-4B. Analgesic effects of KP were dose-dependent in both pain models. FIG. 4A shows that systemic KP is analgesic for nerve constriction injury pain. Overview of timeline of systemic injection and behavioral metrics is shown in the bottom panel. Mice were injected intraperitoneal (i.p.) with either 10 mg/kg or 30 mg/kg KP daily after nerve constriction injury (PSNL). It is shown that analgesic effects of KP for sensitized mechanical withdrawal were dose-dependent, namely less accentuated at 10 mg/kg and more pronounced at 30 mg/kg (n=6-7 for KP treatment, n=13 for vehicle control; *p<0.05, ***p<0.001, ****p<0.0001, mixed model statistics). FIG. 4B shows that systemic KP reduces mechanical allodynia in bone cancer pain evoked by infusion of mouse LLC lung cancer cells into the femur. Overview of timeline of systemic injection and metrics is shown in the bottom panel. Mice were injected i.p. with either 10 mg/kg or 30 mg/kg KP daily. It is shown that analgesic effects of KP for sensitized mechanical withdrawal were significant at Day 10 and 14 for 30 mg/kg when compared to 10 mg/kg of KP or compared to vehicle control (n=5 mice/group; *p<0.05, **p<0.01, two-way ANOVA).

In bone cancer pain, there was no significant analgesic effect of KP at 10 mg/kg, but significant behavioral effects of KP at 30 mg/kg (FIG. 4B). Importantly, protracted analgesia was observed, e.g. an almost complete elimination of pain hypersensitivity after nerve constriction on Day 7-14. The effects on bone cancer pain were also protracted and became apparent at Day 10 and Day 14, only at the higher dose of 30 mg/kg KP.

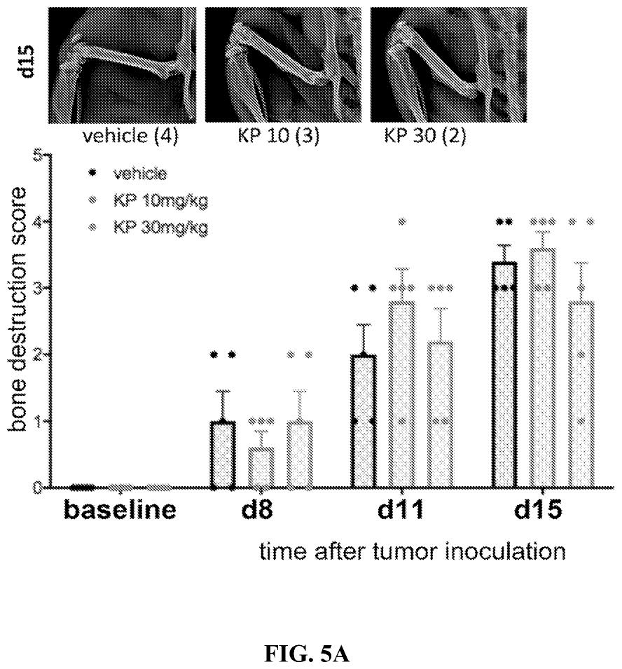

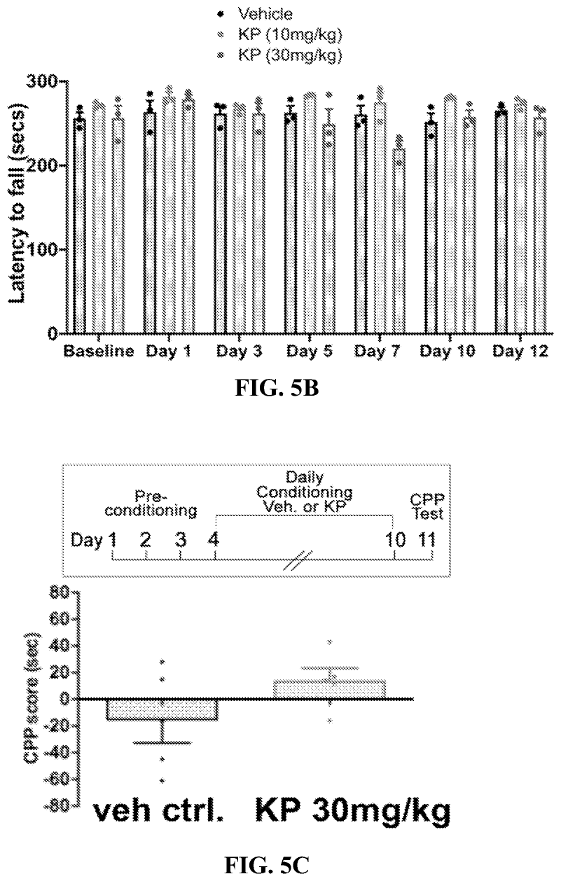

This particular time-course can be interpreted as re-programming of sensitized nociception. These findings are rather not suggestive of selective analgesic inhibition of a pro-algesic ion channel or receptor. Thus, in-vivo, KP functions as an analgesic when administered systemically, in both neuropathic pain model and a bone cancer pain model. Of note and in contrast to its effective analgesic profile in bone cancer pain, KP did not significantly inhibit osteolysis/bone damage (FIG. 5A), indicating that it did not contain growth of the implanted mouse lung carcinoma cells.

Further study was done to determine whether the analgesic effects of KP were mediated centrally at the spinal cord level. KP was injected intrathecally (i.t.; 30 μg) in mice with nerve constriction injury. Reduced mechanical allodynia was observed, using the CCI method of nerve constriction (FIG. 4C). That is, intrathecal (i. t.) KP reduced mechanical allodynia in neuropathic pain following nerve constriction injury. The overview of timeline of nerve constriction (CCI), i. t. injection and behavioral metrics is shown in the bottom panel. The analgesic effects of KP for sensitized mechanical withdrawal were observed upon daily i. t. injection (30 μg) (bar diagram). Increasing sensitization in vehicle injected controls was observed. Withdrawal behavior was measured at two times after i. t. injection, +3 h, +24 h (*p<0.05, n=5 mice/group, *p<0.05, **p<0.01, ****p<0.0001, two-way ANOVA). The analgesic effect was apparent at 3 h and persisted at 8 h. This time-course is in keeping with the rapid effect on Kcc2 gene regulation that was observed in primary cortical neurons (FIGS. 3A-3C).

In order to learn whether the i. t. analgesic effect of KP relies on KCC2 transporter-function, KP was co-applied with the KCC2 chloride transport inhibitor, VU0240551 (30 μg). The results show that the co-application of KP and VU240551 blocked the central analgesic effects of KP (FIG. 4D). Behavior assays were conducted at 3 h, 4 h and 5 h on Day 7 after i.t. injection (n=5 mice/group; **p<0.01, ***p<0.001, two-way ANOVA). The observed elimination of the i. t. analgesic effects of KP suggests that the central analgesic effect of KP depended on KCC2-mediated chloride extrusion.

In view of these beneficial effects of KP on pathologic pain, KP was evaluated next to determine whether it had undesirable effects on the CNS in terms of sedation, impairment of motor stamina, balance and coordination in preparation for translation toward clinical use. First, the results show that KP did not contain growth of mouse LLC lung tumor cells infused into the femur (FIG. 5A). Osteolytic damage to femur was not contained at any time point, e.g., not at 10 mg/kg or at 30 mg/kg KP (n=5 mice per group). Next, effect of KP on motor function and coordination of mice was evaluated (i.e., rotarod assay). Rotarod testing of KP-treated mice (10 mg/kg, 30 mg/kg; i.p.) showed that KP did not induce unwanted side effects (FIG. 5B) in that it did not affect motor stamina and coordination in mice, with the one-time exception of high-dose KP on Day 7 (n=3-4 mice per group).

Further KP (30 mg/kg; i.p.) was evaluated to determine whether it can trigger brain reward mechanisms by measuring conditioned place preference (CPP). The results show that KP did not evoke CPP in that no significant difference in CPP scores between KP and vehicle was observed (FIG. 5C) (timeline for CPP assay was shown on top; n=5 mice/group). Thus, KP functions as an analgesic in relevant preclinical mouse models and does not cause unwanted effects including effects on reward, sedation, lack of coordination, and reduced stamina. In pathologic pain, KP acts centrally to mediate analgesic effects, and this involves KCC2 chloride extrusion.

### Example 3: Renormalization of E_{GABA} in Spinal Cord Dorsal Horn

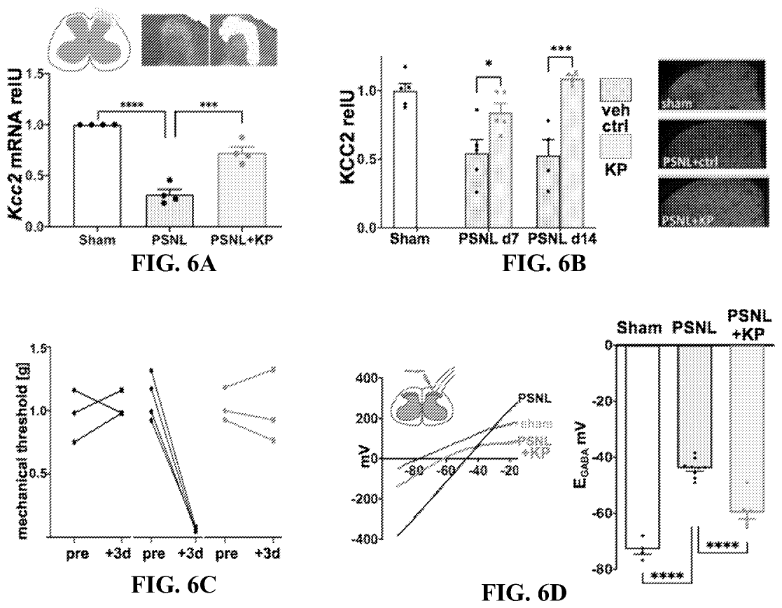

With the above findings and in view of the spinal cord dorsal horn (SCDH) as the likely site of analgesic action of kenpaullone (KP), the study was then focused on SCDH Kcc2 expression and function in nerve injury and response to KP. It was found that in the SCDH, KP repaired attenuated Kcc2 expression caused by partial sciatic nerve ligation (PSNL), at both the mRNA and protein levels (FIGS. 6A-6B). To measure Kcc2 mRNA, SCDH Rexed-layers I-II was micro-dissected with laser capture followed by RT-qPCR (FIG. 6A). Lamina-I/II area of spinal cord dorsal horn (SCDH) is highlighted in the top left. Image representation of Lamina-I/II area before and after laser capture microdissection is shown in the top right. The bar diagrams show that KP (10 mg/kg) rescued significantly attenuated Kcc2 mRNA expression in the SCDH after nerve constriction injury (PSNL) compared to vehicle treated mice (n=4 mice/group, ****p<0.0001, one-way ANOVA).

SCDH KCC2 protein expression was measured morphometrically after KCC2 immunolabeling using SCDH Laminae I-II as the region-of-interest (FIG. 6B). The results show that KP (10 mg/kg) increased KCC2 protein expression in SCDH compared to vehicle control in PSNL nerve constriction injury. Representative KCC2 immuno-staining of the SCDH in nerve constriction injury (PSNL) is shown in the right hand panels (n=5 mice/group, *p<0.05; ***p<0.001, one-way ANOVA).

Nerve injury-induced downregulation of KCC2 has been shown to cause chloride reversal potential change in SCDH neurons after application of GABA (E_{GABA}) in spinal cord slices. The further study investigated whether repair of attenuated Kcc2 expression was associated with re-normalization of E_{GABA}. Prior to interrogation of spinal cord slice preparations at 72 h post-injury, robust mechanical allodynia of young mice by peripheral nerve constriction injury (PSNL) and its almost complete behavioral reversal by systemic treatment with KP at 30 mg/kg demonstrated (FIG. 6C). It was noted that PSNL injury sensitized the juvenile mice more than adults. Also, behavioral rescue with KP was very potent in juveniles. Next, in spinal cord slice preparations derived from these animals, SCDH lamina-II neurons were investigated by perforated patch-clamp (FIG. 6D). Spinal cord slices were isolated from the same juvenile mice as shown in FIG. 6C, and lamina-II neurons were examined for E_{GABA} using the perforated patch method, illustrated by the schematic, 1-3 neurons per mouse. The left-hand panel shows a representative I-V plot. The right-hand bar diagram shows quantification of E_{GABA} indicating a significant depolarizing shift in sham vs PSNL with vehicle treatment (“PSNL”). Significant hyperpolarization in response to KP treatment was observed in PSNL mice. Of note, PSNL plus KP was not different from sham injury (n=4 neurons (sham), n=9 neurons (PSNL), n=6 neurons (PSNL+KP); ****p<0.0001, one-way ANOVA).

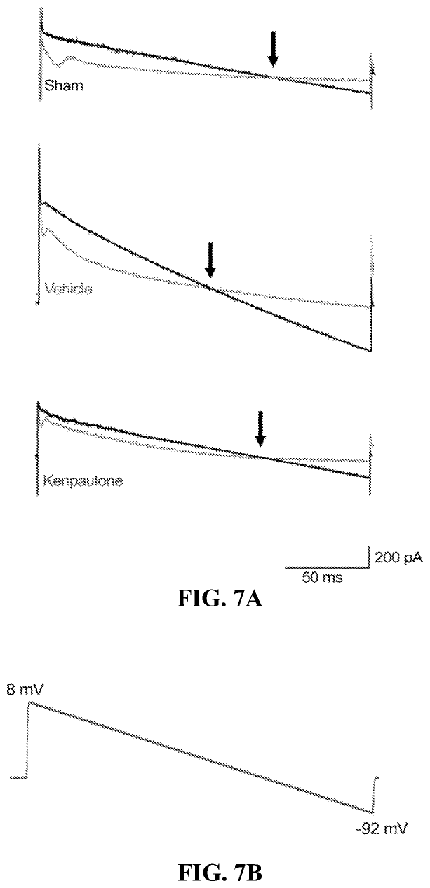

Electrophysiological recordings from SCDH are shown in FIGS. 7A-7B. Current responses of layer-II neurons to a voltage ramp from +8 to −92 mV are shown in FIG. 7A in control group (grey traces, obtained before GABA puff), or at the end of a puff of GABA (black traces) in sham, PSNL+vehicle and PSNL+KP groups. Reversal potential of the GABA-evoked current is at the voltage where the grey and black traces intersect (arrow). FIG. 7B is a voltage ramp from +8 to −92 mV.

The data show that the more positive, thus more excitable E_{GABA} reversal potential in PSNL mice was significantly lowered toward more negative values in KP-treated animals (e.g., FIG. 6D). This documentation of re-normalization of E_{GABA} in SCDH lamina-II neurons is consistent with repair of Kcc2 expression in SCDH in KP-treated mice. Thus, in a constriction nerve injury model in mice, the central analgesic action of KP relies on the enhanced gene expression of Kcc2 and re-normalization of E_{GABA} in pain-relaying neurons in the SCDH.

### Example 4: Cellular Mechanism of Action of Kenpaullone in Central Neurons

The above findings set up a compelling rationale to deconstruct the cellular mechanism of action of kenpaullone (KP) that accounts for its effects in SCDH neurons and thus its sensory effects. These studies were conducted in primary cortical neurons because 1) these neurons were used for the initial screen, 2) there was rodent—human similarity in terms of the effects of KP on Kcc2/KCC2 gene expression, and 3) the effects of KP on Kcc2 gene expression and KCC2 chloride transporter function in these neurons were highly similar to the findings in SCDH lamina-II neurons, which cannot be cultured as readily for mechanistic cellular studies.

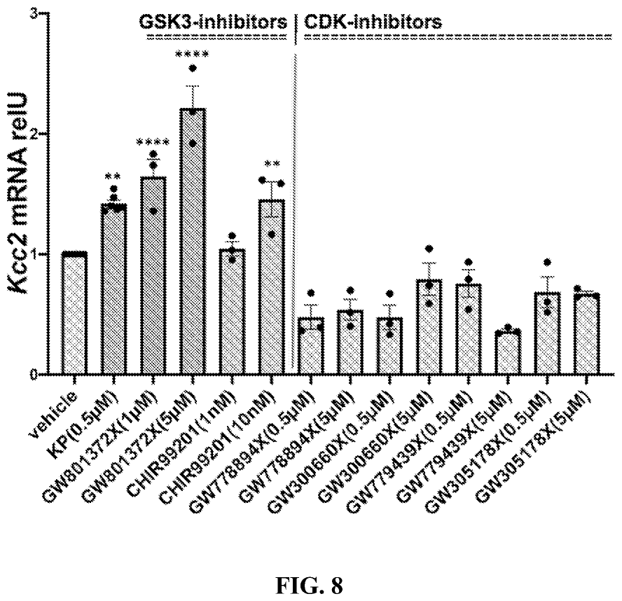

First, the question whether KP inhibiting GSK3β or CDKs is responsible for increasing expression of Kcc2 was addressed. Using a set of GSK3 inhibitors different from KP, it was demonstrated increased expression of Kcc2. In contrast, a suite of CDK-inhibitory compounds did not increase (but decreased) expression of Kcc2 (FIG. 8). As shown therein, GSK3-inhibitors increased Kcc2 mRNA expression, measured by RT-qPCR, in a dose-dependent manner, whereas several CDK-inhibitors did not increase Kcc2 mRNA expression, or rather, reduced Kcc2 mRNA expression. Rat primary cortical neurons were used. Results represent the average mRNA expression of 3-6 independent neuronal cultures (**p<0.01, ****p<0.0001, compound vs vehicle, one-way ANOVA). This suggests that KP increases Kcc2 expression in central neurons by inhibiting GSK3β, not CDKs.

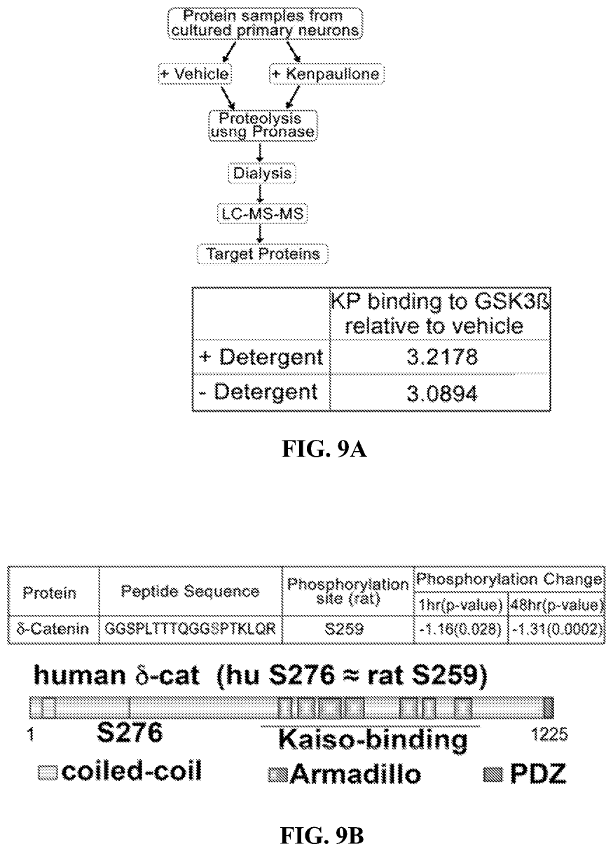

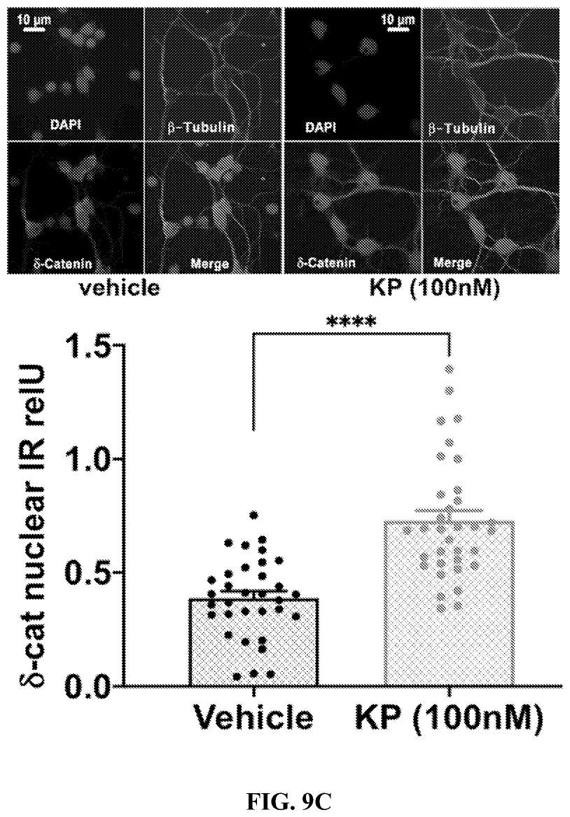

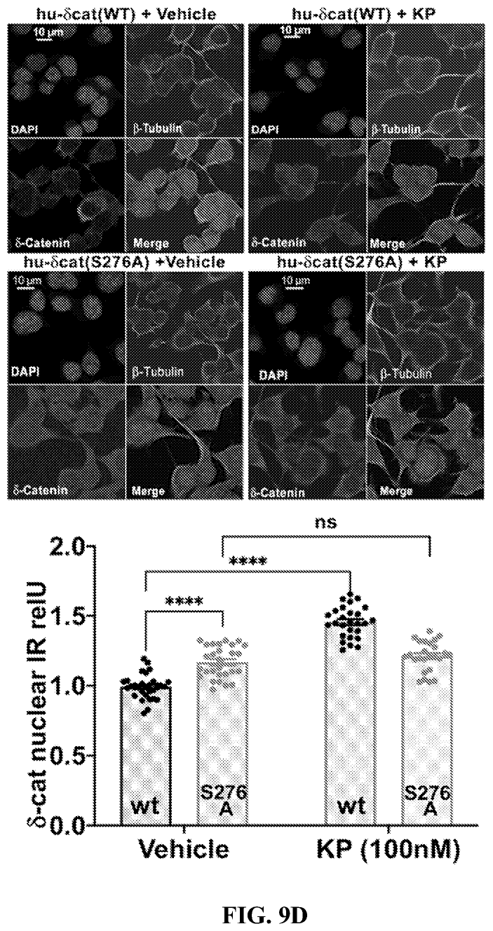

Next, a drug affinity responsive target stability (DARTS) assay was then conducted to study direct binding of KP to GSK3β in primary cortical neurons (FIG. 9A). As shown therein, DARTS methodology was used to identify proteins that bind to KP in rat primary cortical neurons (top panel). Also shown is that KP binding to GSK3β is independent of detergent treatment of the protein sample prepared from the neuronal culture (bottom panel). It is noted that binding to GSK3β was documented whereas binding to CDKs was not. The affirmative findings are consistent with previous reports in cell lines yet novel in primary neurons. Of note, other known kinase targets of KP, such as CDKs, were not identified in the unbiased DARTS assay. This confirms the result with inhibitor compounds that it is the GSK3β-inhibitory function of KP that is responsible for its Kcc2/KCC2 gene expression enhancing property in neurons.

Further study was done to identify kinase targets of GSK3β that are differentially phosphorylated in response to KP. Using unbiased phosphoproteomics assays in rat primary cortical neurons, the neuronal catenin, δ-catenin (CTNND2; δ-cat) was identified. It was discovered that the serine at position 259 was one site at which differential phosphorylation occurred short-term (1 h) and persisted long-term (24 h) in response to KP (FIG. 9B). The respective residue in human δ-cat is S276. β-catenin (β-cat) could also be a GSK3β kinase target, yet β-cat was not significantly differentially phosphorylated. δ-cat (S276) has been previously described (Herskowitz, et al., 2010, J Proteome Res 9: 6368-6379), but its identification in primary neurons is novel. Catenin phosphorylation is known to facilitate its own intracellular degradation via ubiquitination (Orford, et al., 1997, The Journal of Biological Chemistry 272: 24735-24738; Oh, et al., 2009, The Journal of Biological Chemistry 284: 28579-28589). To examine whether non-phosphorylated δ-cat traffics to the neuronal nucleus, specific δ-cat immunolabeling followed by confocal microscopy and morphometry was conducted. The results indicate that KP treatment of primary neurons enhanced δ-cat nuclear transfer (FIGS. 9C-9D). Representative immuno-labeling of β_{III}-tubulin (neuronal isoform of β-tubulin) and δ-cat before and after KP treatment in rat primary cortical neurons is shown in FIG. 9C, top panel. DAPI stain was used to define nuclear compartment for morphometric assessment, i.e., DAPI is the counterstain for nuclei. The bar diagram illustrates that KP significantly increased δ-cat nuclear translocation (FIG. 9C, bottom panel) (n=33-34 neurons per group, ****p<0.001 KP vs. vehicle, t-test). Representative immuno-labeling of β_{III}-tubulin and δ-cat before and after KP treatment in differentiated N2a mouse neural cells which were transfected with either hu-δ-cat (WT), hu-δ-cat (S276A) or control vector is shown in FIG. 9D, top panel. The bar diagram illustrate that KP significantly enhanced nuclear transfer of δ-cat when transfected with δ-cat (WT) (FIG. 9D, bottom panel). Mutation δ-cat (S276A) increased nuclear transfer, but treatment with KP had no effect (n=25-30 neurons/group ****p<0.0001 vs. vehicle, mixed effects statistics).

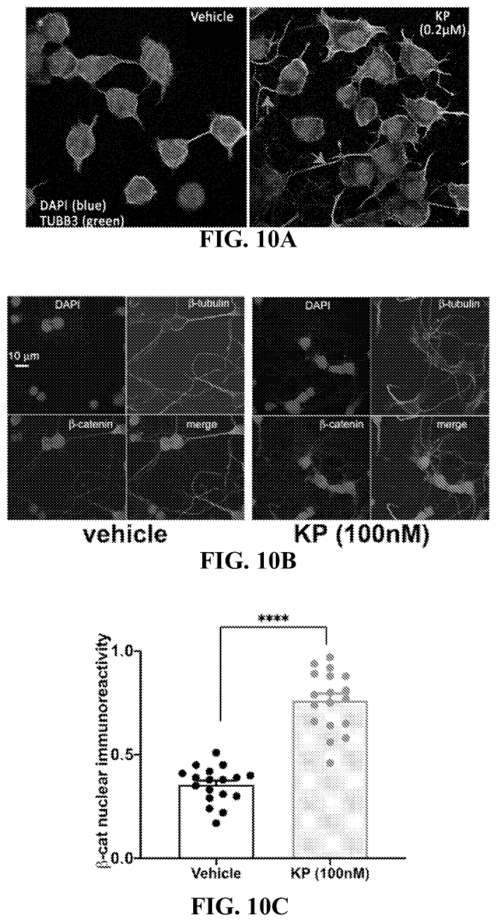

Given the known binding of β-cat/δ-cat, β-cat was immune-labeled and a similar result was obtained (FIGS. 10A-10C). Neuronalization of N2a cultured cells in response to KP (0.2 μM for 3 days) was conducted and increased process formation and expression of neuronal β_{III}-tubulin was observed on micrograph images (FIG. 10A; green, red arrows). DAPI blue stain is used to define nuclear compartment for morphometric assessment. Representative immuno-labeling of neuronal β_{III}-tubulin (green) and β-catenin (red) before (left-hand panels) and after KP treatment (right-hand panels) in primary cortical neurons from rat is shown in FIG. 10B. The results show that KP significantly increased β-catenin translocation into the nucleus (FIG. 10C; n=18 neurons per group), thus corroborating the δ-cat nuclear transfer results. That is, KP enhances neuronal differentiation and facilitates β-catenin translocation to the nucleus.

To investigate the relevance of δ-cat residue S276, N2a neural cells were studied because these cells transfect at higher efficiency than primary cortical neurons. In the cultures used for this study, N2a cells expressed neuronal β_{III}-tubulin in elongated processes (FIGS. 10A-10C) as an indicator of their neuronal differentiation. Furthermore, nuclear transfer of δ-cat was significantly enhanced upon KP treatment in N2a cells transfected with human δ-cat (WT) (FIG. 9D). This increase in nuclear transfer was very similar to the trafficking that was recorded in primary cortical neurons, thus validating the cell line. To determine the function of δ-cat residue S276, δ-cat (S276A) was transfected, which resulted in significantly increased nuclear transfer of δ-cat (S276A), using morphometry. Of note, there was no increase of nuclear transfer of δ-cat (S276A) upon KP treatment (FIG. 9D). Thus, it is suggested that δ-cat (S259/S276) (rat/human) is very likely a relevant phosphorylation site in δ-cat and a GSK3βkinase target in neurons. Inhibition of GSK3β or rendering S276 phosphorylation-resistant enhances nuclear transfer of δ-cat, also of its binding partner β-cat. It is concluded that catenins enhance Kcc2 gene expression in neurons.

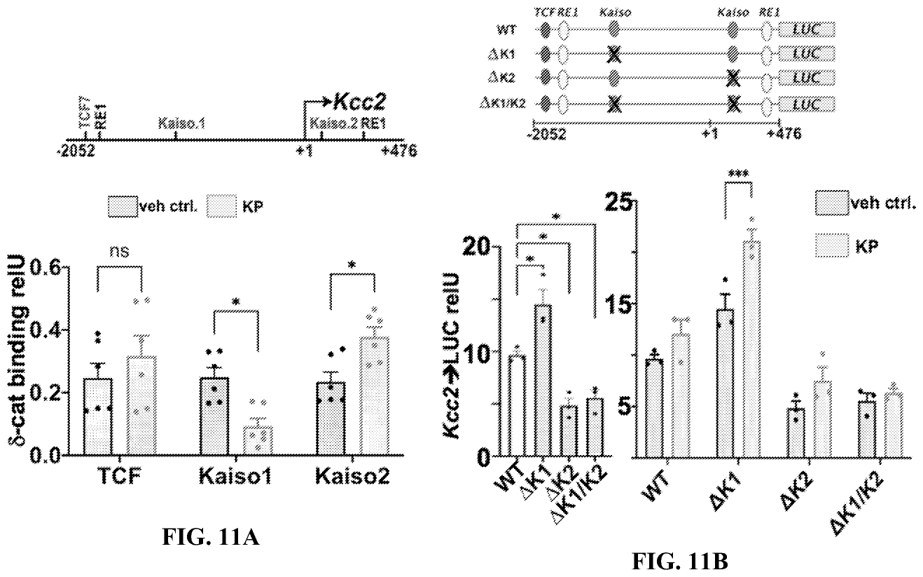

Consequently, effects of catenins on the Kcc2 promoter was explored. Two Kaiso binding sites (potential sites for δ-cat) (Rodova, et al., 2004, Mol Cell Biol 24: 7188-7196; Dai, et al., 2011, Cancer Science 102: 95-103) were identified in the Kcc2 proximal promoter using computational methods (Aerts, et al., 2005, Nucleic Acids Res 33: 393-396). The two Kaiso binding sites bracketed the transcriptional start site (TSS) of the Kcc2 gene (FIG. 11A, top panel). In rat primary cortical neurons, δ-cat was bound to both Kaiso sites in the Kcc2 promoter (FIG. 11A). The structure of mouse Kcc2 gene encompassing 2.5 kb surrounding the transcription start site (TSS; +1) is shown therein (top panel), along with the location of DNA binding sites: Kaiso1 (−1456 to −1449), Kaiso2 (+83 to +90) and TCF (−1845 to −1838) relative to TSS, all three sites can bind δ-cat via Kaiso (Kaiso1, 2 sites) and β-cat (TCF). Chromatin immuno-precipitation (ChIP) using anti-δ-cat antibody in rat primary cortical neurons reveals binding of δ-cat to all three sites (bottom panel). KP treatment significantly increased binding of δ-cat to the Kcc2 promoter on the Kaiso2 binding site, significantly reduced binding to Kaiso1, and non-significant increase at TCF (n=6 independent neuronal cultures were subjected to ChIP; *p<0.05 KP-treatment vs vehicle, t-test).

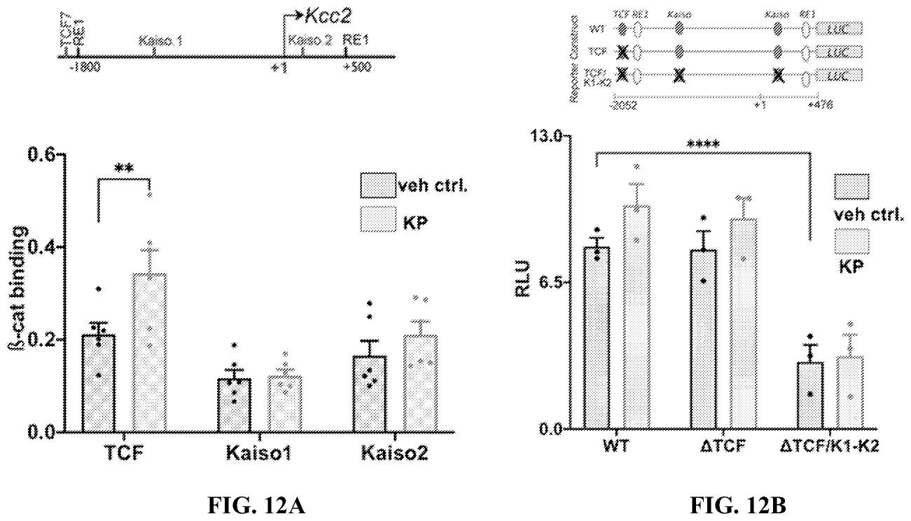

Interestingly, treatment with KP inhibited the binding of δ-cat to the upstream site. Binding of δ-cat to the site 3′ to the TSS was enhanced, suggesting that the upstream site functions as repressor and the 3′ site as enhancer. β-cat bound to a TCF (T-cell factor) DNA-binding site close to the 5′ RE-1 site within the Kcc2 promoter (FIGS. 12A-12B), and treatment of cells with KP significantly increased this interaction, indicative of enhancement.

Promoter expression constructs were created with rationally-targeted deletions to interrogate the effects of the Kaiso- and TCF-binding sites on activity of the Kcc2 promoter and to determine if this activity was regulated by KP. For ease of transfection, key to this method, N2a neural cells were again used. The 5′ and 3′ δ-cat Kaiso binding sites functioned in repressive and enhancing manners, respectively (FIG. 11B). Mouse Kcc2 promoter constructs are shown in the top panel, Kaiso1, -2 were deleted and a ΔK1/K2 construct was built devoid of both sites. Dual-RE1 sites and TCF site shown for orientation, also TSS at +1. Luciferase (LUC) activity of Kcc2 promoter constructs in N2a cells with neuronal differentiation (bar diagram on the bottom). ΔK1 significantly increased over WT, AK2 and ΔK1/K2 significantly decreased vs. WT (left hand, n=3 independent culture per transfection, *p<0.05, 1-way ANOVA). Shown in right-hand is repressive Kaiso1 binding site deletion with enhanced activity of the Kcc2 promoter, which was further significantly enhanced in response to KP (n=3 independent cultures per transfection; ***p<0.001 KP-treatment vs. vehicle for the respective construct, t-test).

Further study shows that presence of the 3′ δ-cat Kaiso binding site and absence of the 5′ site led to significantly enhanced activity of the Kcc2 promoter upon treatment with KP. Deletion of both Kaiso sites led to markedly reduced promoter activity and non-responsiveness of the construct to KP. Deletion of the TCF binding-site from the Kcc2 promoter did not change Kcc2 promoter activity or its response to KP treatment (FIGS. 12A-12B). Shown in FIG. 12A, KP increased β-cat binding to the TCF site in the Kcc2 promoter. The structure of mouse-Kcc2 gene encompassing 2.5 kb surrounding the TSS (+1) is shown in the top panel. As shown in the bar diagram on the bottom, Chromatin immuno-precipitation using anti-β-cat antibody in primary rat cortical neurons revealed binding of β-cat to TCF, to minor degree to Kaiso1, 2. KP treatment significantly increased binding of β-cat to the Kcc2 promoter on the TCF binding site (n=6 independent rat primary neuron cultures were subjected to ChIP, **p<0.01 t-test, KP-treated vs. vehicle). However, the triple-deletion of both Kaiso sites and the TCF site rendered the construct minimally active and completely non-responsive to KP. Relevance of TCF DNA binding site on regulation of Kcc2≥LUC is shown in FIG. 12B. N2a differentiated cells were used for this study. Mouse Kcc2 promoter constructs are shown in the top panel. The bar diagram on the bottom shows that KP did not increase promoter activity for any of the constructs. Triple-deletion of TCF and both Kaiso sites rendered the Kcc2 promoter very low in activity, compared with WT (without KP), at <⅓ of its activity, which is less than the ΔΔKaiso1,2 deletion construct (FIG. 11B; <½) (n=3 independent cultures, ****p<0.0001 WT vs triple-deletion construct, 1-way ANOVA).

These data suggest that δ-cat, a kinase target of GSK3β in CNS neurons, traffics to the nucleus increasingly upon GSK3β inhibition. In the nucleus, δ-cat interacts with the Kcc2 promoter to enhance Kcc2 expression via two Kaiso DNA-binding sites. β-cat co-traffics to the nucleus with δ-cat in response to KP. β-cat is not a significant neuronal GSK3β kinase target. It plays an ancillary role in enhancement of Kcc2 gene expression.

### Example 5: Synergistic Effects of Kenpaullone and Paclitaxel

Two commonly used breast cancer cell lines (MCF7 and MDA MB-231) and one melanoma cell line (A375) were used in this study. 100 nM Kenpaullone (KP) and/or 1 nM or 10 nM paclitaxel (Taxol) were administered to the cell lines to test their impact on cell viability. Each bar represents an average of 6 experiments, and error bars represent SEM. Cell line maintenance and MTT cell viability assay were done according to Xiao et al. (2018, BMC Cancer, 18(1): 675). Cell viability was assessed after 72 hours.

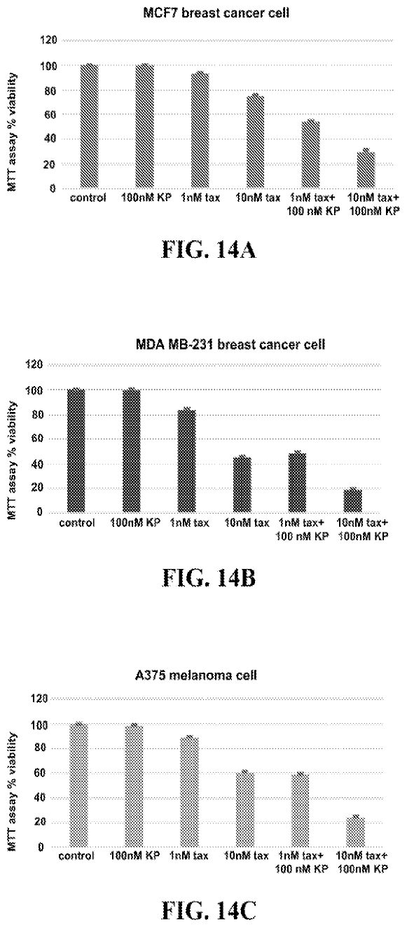

The results show that KP alone did not influence cell viability at 100 nM; however, when combined with 1 nM or 10 nM paclitaxel, the cell viability was reduced robustly and highly significantly (FIGS. 14A-14C). It was known that the tested cell lines were sensitive to 1 nM and 10 nM paclitaxel, which was also demonstrated in this study.

Overall, this study suggests that KP and paclitaxel function synergistically in their impact on cell viability of two commonly used breast cancer cell lines and a melanoma cell line.

### Example 6: Effects of S-Catenin Overexpression on Cell Proliferation and Differentiation

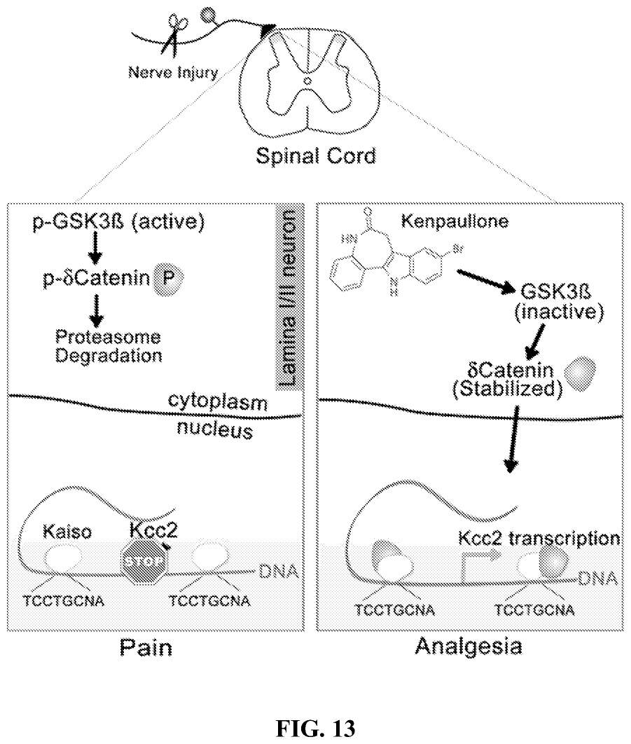

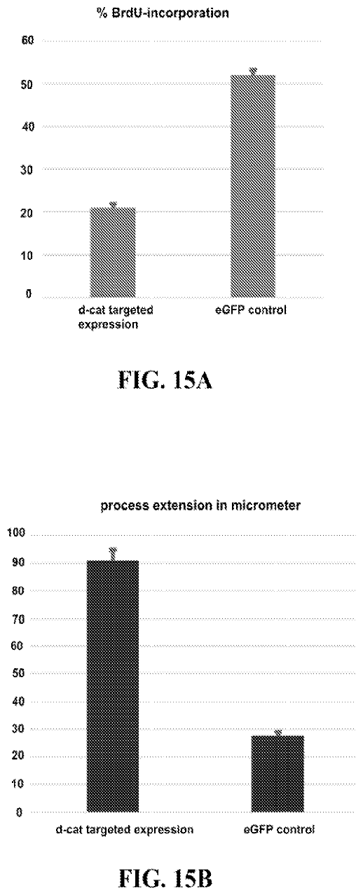

Targeted overexpression of δ-catenin was evaluated in neuronal-neuroblastoma cell line N2a as described in FIG. 13A. N2a Cell line was maintained according to the section “Materials and Methods” above. BrdU measurements were done according to Titus et al. (2017, Journal of Neuroscience 26: 3612-3616). For % BrdU incorporation data, each bar represents an average of 6 experiments and errors bars represent SEM (FIG. 15A). For process extension data, the length measurements of N2a cells' processes (n=36 neuronal cells/group) were done according to Williams et al. (2016, Molecular Biology of The Cell, 27(3): 518-534) (FIG. 15B). Assays were conducted 4 days after transfection.

The results show significantly reduced % BrdU incorporation (FIG. 15A) and significantly enhanced neuronal process extension (FIG. 15B) in cultures with δ-cat targeted expression. The reduced % BrdU incorporation suggests that δ-catenin overexpression reduced cell proliferation, and the enhanced neuronal process extension suggests that δ-catenin overexpression enhanced cell differentiation.

### DISCUSSION

Evidence that reduced expression of the neuronal chloride extruding transporter, KCC2, critically contributes to chronic pathologic pain led to the effort of seeking compounds that could increase Kcc2/KCC2 gene expression. Using measures of Kcc2 promoter activity, Kcc2 mRNA abundance, and [Cl-]i, an unbiased screen was conducted in primary cortical neurons of two NCI libraries containing 1057 compounds that inhibit growth of malignantly-transformed cells. This screen identified kenpaullone (KP) among others, which is a GSK3/CDK kinase inhibitor with neuroprotective properties (Liu et al., 2016, Cell reports 14: 115-128; Reinhardt et al., 2019, Stem Cell Reports 12: 502-517; Schultz et al., 1999, J Med Chem 42: 2909-2919, Skardelly et al., 2011, Neuroscience 29: 543-547; Yang et al., 2013, Cell Stem Cell 12: 713-726; Zaharevitz et al., 1999, Cancer research 59: 2566-2569). These studies of KP revealed at least the following principal findings: 1) KP directly enhanced Kcc2/KCC2 gene expression (not KCC2 transporter function) in a concentration-dependent manner and lowered [Cl-]i in cultured mouse, rat, and human neurons; 2) systemic administration of KP to mice attenuated constriction nerve injury pain and bone cancer pain in preclinical models in a dose-dependent manner; 3) intrathecal administration of KP to mice attenuated constriction nerve injury pain depending on spinal KCC2 chloride transporter activity; 4) systemic administration of KP to mice with constriction nerve injury repaired defective Kcc2 gene expression in SCDH neurons and shifted the GABA-evoked chloride reversal potential to more negative and electrically stable measures; and 5) the mechanism by which KP enhanced Kcc2 gene expression was by binding to and inhibiting GSK3β, inhibiting phosphorylation of δ2-cat at position S259 in rat (or S276 in human), which increased nuclear transfer of δ2-cat. In the nucleus, δ2-cat bonded to and enhanced the Kcc2 promoter via two Kaiso-binding sites surrounding the TSS of the Kcc2 gene. To test whether this mechanism elucidated in neural cells was also valid in live animals, spinal transgenesis of δ2-cat (S276A) was found to attenuate constriction nerve injury pain in mice. Thus, one can conclude that KP and the new GSK3β->δ2-cat->Kaiso->Kcc2 signaling pathway may represent a strategic bridge-head for therapeutics development for treatment of pathologic pain. Beyond pain, this could also apply to other neurologic and mental health conditions in which restoration of KCC2 function is important, such as epilepsy, traumatic spinal cord injury, traumatic brain injury, neurodegeneration and neurodevelopmental disorders. The proposed analgesic mechanism is summarized in FIG. 13.

Compounds from two NCI libraries that are capable of interfering with gene regulation in CNS neurons were identified. Non-dividing CNS neurons such as primary developing cortical neurons enabled identification of compounds that enhanced the Kcc2 promoter, among which kenpaullone (KP) was selected as one of the “winner” compounds for in-depth exploration. Though KP is predicted to have multiple targets in CNS neurons, the data shown herein provide evidence that KP binds to neuronal GSK3β and not to CDKs. In addition, it is known that KP inhibits GSK3β with highest potency from amongst known targets (Knockaert et al., 2002, The Journal of Biological Chemistry 277: 25493-25501; Kunick et al., 2004, Bioorg Med Chem Lett 14: 413-416; Schultz et al., 1999, J Med Chem 42: 2909-2919). The present disclosure also demonstrated that GSK3β inhibition by KP directly upregulated Kcc2 gene expression via the δ-catenin-Kaiso pathway. This mechanism of a GSK3-inhibitory compound has not been reported previously. Of note, this novel concept held true in primary human neurons in which KP also enhanced synaptic maturation. The respective marker, synaptophysin, showed remarkable co-localization with upregulated KCC2 protein. Although KP can inhibit other kinases, the data disclosed herein suggest that inhibition of GSK3β and subsequent enhancement of KCC2 gene expression via δ-catenin are very important perhaps dominant mechanisms of action of KP as it attenuates pathologic pain. Additionally, s-cat-Kaiso likely affects multiple neuronal genes, but the present disclosure suggests that enhanced KCC2 gene expression and KCC2 function are the major analgesic effector mechanisms of KP. Another argument, mechanistically weaker but translationally relevant, is the absence of unwanted effects of Kcc2 expression-enhancing strategy on choice behavior, motor stamina, and coordination. Effective targeting of multiple pathways would likely impact these behaviors. However, the behavioral profile for KP was similarly benign as that of KCC2 chloride-extrusion enhancing compounds.

The screening strategy disclosed herein addressed a fundamental problem of pathological pain. This vastly unmet medical need, rooted in its chronicity and overall debilitating impact, is driven by genetic reprogramming, which results in a maladaptive phenotype (Bai et al., 2015, Translational Research: the Journal of Laboratory and Clinical Medicine 165: 177-199; Doyon et al., 2013, Expert Review of Neurotherapeutics 13: 469-471; Kuner, 2010, Nat Med 16: 1258-1266; Liang et al., 2015, Epigenomics 7: 235-245; Sommer, 2016, Science 354: 588-592). One very important mechanism to contribute to the maladaptive phenotype is attenuated expression of Kcc2 because of its relevance for inhibitory transmission in pain-relevant neural circuits (Coull et al., 2003, Nature 424: 938-942; Gagnon et al., 2013, Nat Med 19: 1524-1528; Kahle et al., 2014, JAMA Neurology 71: 640-645; Li et al., 2016, Cell Reports 15: 1376-1383; Mapplebeck et al., 2019, Cell Reports 28: 590-596 e594; Price et al., 2005, Curr Top Med Chem 5: 547-555). This has also been postulated in other pathologic conditions of the CNS as a general pathogenic feature. Regulation of Kcc2 gene expression by GSK3β and its kinase target δ-cat is a novel insight of the present disclosure. This concept permits rational exploration of links between GSK3β->δ-cat and attenuated Kcc2/KCC2 gene expression and the resulting malfunction of inhibitory neurotransmission in several other relevant neurologic and psychiatric conditions, such as epilepsy, traumatic brain/spinal cord injury, Rett Syndrome, Autism Spectrum Disorders, and perhaps also Alzheimer's Disease and other neurodegenerative diseases. KP was selected because of its previously reported neuroprotective properties for spinal motoneurons, brainstem auditory relay neurons, and hypoxia-injured hippocampal neurons (Liu et al., 2016, Cell Reports 14: 115-128; Reinhardt et al., 2019, Stem Cell Reports 12: 502-517; Skardelly et al., 2011, Neuroscience 29:543-547; Teitz et al., 2018, The Journal of Experimental Medicine 215: 1187-1203; Winkelmann et al., 2015, Cell Death & Disease 6:e1776; Yang et al., 2013, Cell Stem Cell 12: 713-726). Neuroprotective properties for a novel analgesic are important discovery because chronic pain is associated with non-resolving neural injury mediated by neuroinflammation. The repurposing of a GSK3β-inhibitory compound as an analgesic reprogramming agent that upregulates Kcc2/KCC2 expression links chronic pathologic pain to neurodegeneration, at both the basic science and translational neuroscience levels.

Although the present invention and its advantages have been described in detail, it should be understood that various changes, substitutions and alterations can be made herein without departing from the spirit and scope of the invention as defined in the appended claims.

One skilled in the art will readily appreciate that the present invention is well adapted to carry out the objects and obtain the ends and advantages mentioned, as well as those inherent therein. The present examples along with the methods described herein are presently representative of preferred embodiments, are exemplary, and are not intended as limitations on the scope of the invention. Changes therein and other uses will occur to those skilled in the art which are encompassed within the spirit of the invention as defined by the scope of the claims.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference, unless explicitly indicated otherwise. The present disclosure shall control in the event there are any disparities between any definitions and/or description found in the cited references.

1. A method of treating a pathologic pain associated with a malignant growth disorder in a subject in need thereof, the method comprising administering to the subject a therapeutically effective amount of a K+/Cl-cotransporter (Kcc2/KCC2) gene expression enhancer.

2. The method of claim 1, wherein the Kcc2/KCC2 gene expression enhancer is a GSK3 β inhibitor.

3. The method of claim 2, wherein the GSK3β inhibitor inhibits the activity and/or function of GSK3β in a neuronal cell.

4. The method of claim 1, wherein the Kcc2/KCC2 gene expression enhancer is a compound comprising
the general formula (I) (termed NSC180515): 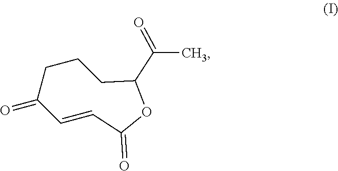
the general formula (II) (termed NSC79456): 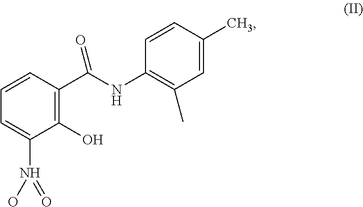
the general formula (III) (termed NSC33006): 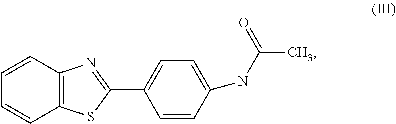
the general formula (IV) (termed Kenpaullone): 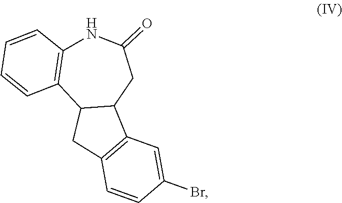
or a pharmaceutically acceptable salt, solvate, hydrate, prodrug, or derivative thereof.

5.-7. (canceled)

8. The method of claim 1, wherein the malignant growth disorder is a cancer.

9. The method of claim 8, wherein the cancer is a bone cancer.

10. The method of claim 1, wherein the malignant growth disorder is a blood cancer, and wherein the blood cancer is multiple myeloma, lymphoma, or leukemia.

11. (canceled)

12. The method of claim 1, further comprising administering to the subject at least one additional therapeutic agent.

13. The method of claim 12, wherein the at least one additional therapeutic agent is a pain killer, a steroid, an anti-cancer drug, a muscle relaxant, an anti-anxiety drug, an antidepressant, an anticonvulsant, an antimetabolite, an antimicrotubule agent, a topoisomerase inhibitor, a cytotoxic agent, a checkpoint inhibitor, or a combination thereof.

14. The method of claim 12, wherein the at least one additional therapeutic agent is NTHES, acetaminophen, lidocaine patches/creams, cisplatin, chlorambucil, procarbazine, carmustine, methotrexate, cytarabine, gemcitabine, vinblastine, paclitaxel, etoposide, doxorubicin, bleomycin, mitomycin, anti-PD1, anti-CTLA4, or a combination thereof.

15.-36. (canceled)

37. A method of restoring KCC2/KCC2 function thereby treating a neurologic and mental health condition in a subject in need thereof, the method comprising administering to the subject a therapeutically effective amount of a K+/Cl-cotransporter (Kcc2/KCC2) gene expression enhancer.

38. The method of claim 37, wherein the condition is a neurodegeneration condition or a neurodevelopmental disorder.

39. The method of claim 37, wherein the condition is epilepsy, a traumatic spinal cord injury, a traumatic brain injury, Rett Syndrome, an Autism Spectrum Disorder, or Alzheimer's Disease.

40. The method of claim 37, wherein the Kcc2/KCC2 gene expression enhancer is a GSK3β inhibitor.

41. The method of claim 40, wherein the GSK3β inhibitor inhibits the activity and/or function of GSK3β in a neuronal cell.

42. The method of claim 37, wherein the Kcc2/KCC2 gene expression enhancer is a compound comprising
the general formula (I) (termed NSC180515): 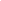
the general formula (II) (termed NSC79456): 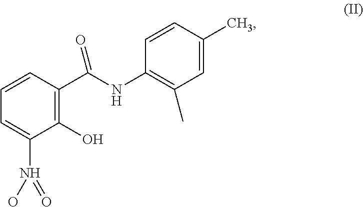
the general formula (III) (termed NSC33006): 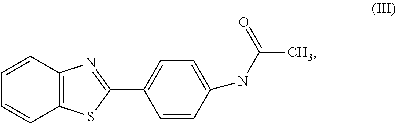
the general formula (IV) (termed Kenpaullone): 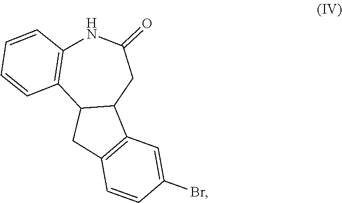
or a pharmaceutically acceptable salt, solvate, hydrate, prodrug, or derivative thereof.

43.-45. (canceled)

46. The method of claim 37, wherein the Kcc2/KCC2 gene expression enhancer is administered intrathecally, intra-cerebroventricularly, intra-cerebrally, perispinally, intra-spinally, intravascularly, intravenously, orally, enterally, rectally, pulmonarily, via inhalation, nasally, topically, transdermally, buccally, sublingually, intravesically, intravitreally, intraperitoneally, vaginally, intrasynovially, intracutaneously, intraarticularly, intraarterially, parenterally, subcutaneously, intrastemally, intralesionally, intramuscularly, intravenously, intradermally, transmucosally, or sublingually.

47.-55. (canceled)

56. The method of claim 37, further comprising administering to the subject at least one additional therapeutic agent.

57. The method of claim 56, wherein the at least one additional therapeutic agent is a pain killer, a steroid, an anti-cancer drug, a muscle relaxant, an anti-anxiety drug, an antidepressant, an anticonvulsant, an antimetabolite, an antimicrotubule agent, a topoisomerase inhibitor, a cytotoxic agent, a checkpoint inhibitor, or a combination thereof.

58. The method of claim 56, wherein the at least one additional therapeutic agent is NTHES, acetaminophen, lidocaine patches/creams, cisplatin, chlorambucil, procarbazine, carmustine, methotrexate, cytarabine, gemcitabine, vinblastine, paclitaxel, etoposide, doxorubicin, bleomycin, mitomycin, anti-PD1, anti-CTLA4, or a combination thereof.
